(19) United States
(12) Patent Application Publication
(10) Pub. No.: US 2024/0416080 A1
(43) Pub. Date: Dec. 19, 2024

(54) MEDICAL DEVICE AND BENDABLE UNIT

(71) Applicant: CANON KABUSHIKI KAISHA, Tokyo (JP)

(72) Inventors: RYO IWASAWA, Kanagawa (JP); YUSUKE NIIKAWA, Kanagawa (JP); ISAO MATSUOKA, Kanagawa (JP)

(21) Appl. No.: 18/819,945

(22) Filed: Aug. 29, 2024

Related U.S. Application Data
(63) Continuation of application No. PCT/JP2023/005411, filed on Feb. 16, 2023.

(30) Foreign Application Priority Data
Mar. 1, 2022 (JP) 2022-030631

(51) Int. Cl. A61M 25/01

(52) U.S. Cl. CPC A61M 25/0113

(57) ABSTRACT

A medical device includes (i) a linear member connected to a bending section and configured to move in an extension direction, where the linear member includes an engaged portion, (ii) a joining section connected to a drive source, where the joining section includes an elastic member configured to retain the engaged portion and a first rotating body rotatable to a pressing position or a retracted position, and (iii) a second rotating body movable to a first position where the first rotating body is located at the pressing position and a second position where the first rotating body is located at the retracted position. When the first rotating body is located at the pressing position, the engaged portion and the elastic member overlap each other as viewed in the extension direction.

### CROSS-REFERENCE TO RELATED APPLICATIONS

This application is a Continuation of International Patent Application No. PCT/JP2023/005411, filed Feb. 16, 2023, which claims the benefit of Japanese Patent Application No. 2022-030631, filed Mar. 1, 2022, both of which are hereby incorporated by reference herein in their entirety.

### TECHNICAL FIELD

The present invention relates to a medical device including a linear member connected to a bending section.

### BACKGROUND ART

U.S. Patent Application Publication No. 2021/0121051 discloses a medical device in which a connecting shaft including a wire that bends a bending section and a rod connected to the wire is mounted to a connection-receptor body including a driving stage connected to an actuator.

According to the medical device described in U.S. Patent Application Publication No. 2021/0121051, the connecting shaft is linearly inserted into the connection-receptor body along one direction and then is rotated to engage the rod with the driving stage, thus joining the wire to the actuator.

One of the objects of the present invention is to use an elastic member to stably fix a linear member connected to a bending section to a joining section connected to a drive source.

### CITATION LIST

### Patent Literature

PTL 1 U.S. Patent Application Publication No. 2021/0121051, issuing as U.S. Pat. No. 11,730,349.

### SUMMARY OF INVENTION

One of the inventions according to the present application is as follows.

A medical device includes a bending section, a linear member connected to the bending section and configured to move in an extension direction so as to bend the bending section, where the linear member includes an engaged portion, a joining section connected to a drive source, wherein the joining section includes an elastic member configured to retain the engaged portion and a first rotating body rotatable to a pressing position where the elastic member is pressed or a retracted position that is retracted from the pressing position, and a second rotating body movable to a first position where the first rotating body is located at the pressing position or a second position where the first rotating body is located at the retracted position. When the first rotating body is located at the pressing position, the engaged portion and the elastic member overlap each other as viewed in the extension direction so that the engaged portion is restricted from moving relative to the elastic member in the extension direction.

Further features of the present invention will become apparent from the following description of exemplary embodiments with reference to the attached drawings.

### BRIEF DESCRIPTION OF DRAWINGS

FIG. 1 is an overall view of a medical system.

FIG. 2 is a perspective view of a medical device and a support stage.

FIG. 3A illustrates a catheter.

FIG. 3B illustrates the catheter.

FIG. 4A illustrates a catheter unit.

FIG. 4B illustrates the catheter unit.

FIG. 5A illustrates a base unit and a wire drive unit.

FIG. 5B illustrates the base unit and the wire drive unit.

FIG. 5C illustrates the base unit and the wire drive unit.

FIG. 6A illustrates the wire drive unit, a joining device, and a bending drive unit.

FIG. 6B illustrates the wire drive unit, the joining device, and the bending drive unit.

FIG. 6C illustrates the wire drive unit, the joining device, and the bending drive unit.

FIG. 7A illustrates mounting of the catheter unit.

FIG. 7B illustrates mounting of the catheter unit.

FIG. 8A illustrates joining of the catheter unit to the base unit.

FIG. 8B illustrates joining of the catheter unit to the base unit.

FIG. 9 is an exploded view illustrating joining of the catheter unit to the base unit.

FIG. 10A illustrates how a leaf spring retains a retained portion.

FIG. 10B illustrates how the leaf spring retains the retained portion.

FIG. 10C illustrates how the leaf spring retains the retained portion.

FIG. 10D illustrates how the leaf spring retains the retained portion.

FIG. 10E illustrates how the leaf spring retains the retained portion.

FIG. 11A illustrates the fixture of a joining section to a drive wire.

FIG. 11B illustrates the fixture of the joining section to the drive wire.

FIG. 12 illustrates the fixture of the joining section to the drive wire.

FIG. 13 illustrates the fixture of the joining section to the drive wire.

FIG. 14 illustrates the fixture of the joining section to the drive wire.

FIG. 15A illustrates the fixture of the joining section to the drive wire.

FIG. 15B illustrates the fixture of the joining section to the drive wire.

FIG. 16A illustrates the catheter unit and the base unit.

FIG. 16B illustrates the catheter unit and the base unit.

FIG. 16C illustrates the catheter unit and the base unit.

FIG. 17A illustrates the movement of an operating unit.

FIG. 17B illustrates the movement of the operating unit.

FIG. 17C illustrates the movement of the operating unit.

FIG. 18A is a cross-sectional view illustrating the movement of the operating unit.

FIG. 18B is a cross-sectional view illustrating the movement of the operating unit.

FIG. 18C is a cross-sectional view illustrating the movement of the operating unit.

### DESCRIPTION OF EMBODIMENTS

An example of the configuration according to the present invention is described below with reference to the accompanying drawings. Note that the sizes, materials, shapes, and locations of constituent elements described in some embodiments should be changed as appropriate in accordance with the configuration and various conditions of the device of the invention.

### Medical System and Medical Device

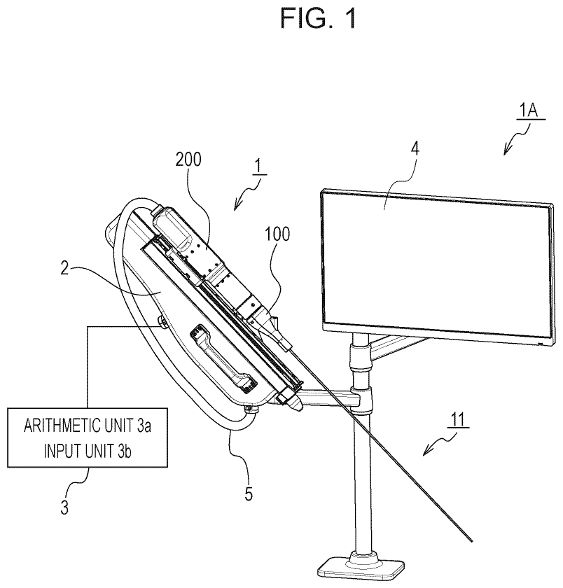

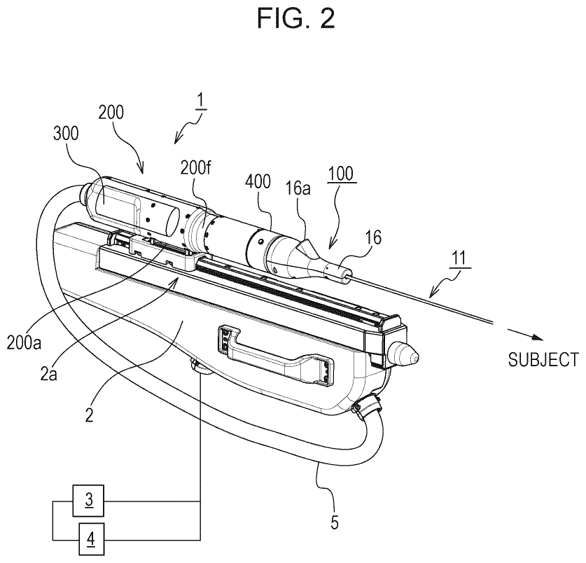

A medical system 1A and a medical device 1 are described with reference to FIGS. 1 and 2. FIG. 1 is an overall view of the medical system 1A. FIG. 2 is a perspective view of the medical device 1 and a support stage 2.

The medical system 1A includes the medical device 1, the support stage 2 to which the medical device 1 is attached, and a control device 3 that controls the medical device 1. According to some embodiments, the medical system 1A includes a monitor 4 serving as a display device.

The medical device 1 includes a catheter unit (bendable unit, bending body unit) 100 including a catheter 11 serving as a bendable body (bending body) and a base unit (drive unit, attached unit, joining unit) 200. The catheter unit 100 is dismountable from the base unit 200.

According to some embodiments, a user of the medical system 1A and the medical device 1 can insert the catheter 11 into the inside of a subject to observe the inside of the subject, collect various specimens from the inside of the subject, and perform procedures on the inside of the subject. According to one embodiment, the user can insert the catheter 11 into the inside of a patient, which is a subject. More specifically, by inserting the catheter into the bronchus via the oral or nasal cavity of the patient, the user can perform procedures such as observing, collecting a sample, and resecting a lung tissue.

The catheter 11 can be used as a guide (sheath) for guiding a medical instrument to perform the above-described procedure. Examples of a medical instrument (a tool) include an endoscopes, forceps, and an ablation device. The catheter 11 itself may function as the above-described medical instrument.

According to some embodiments, the control device 3 includes an arithmetic unit 3a and an input unit 3b. The input unit 3b receives a command and an input for operating the catheter 11. The arithmetic unit 3a includes a storage for storing a program and various data for controlling the catheter, a random access memory, and a central processing unit for executing the program. The control device 3 may also include an output unit that outputs signals for displaying an image on the monitor 4.

As illustrated in FIG. 2, according to some embodiments, the medical device 1 is electrically connected to the control device 3 via a cable 5 that connects the base unit 200 of the medical device 1 to the support stage 2 and the support stage 2. The medical device 1 may be directly connected to the control device 3 by a cable. The medical device 1 may be wirelessly connected to the control device 3.

The medical device 1 is removably attached to the support stage 2 via the base unit 200. More specifically, the medical device 1 is attached such that an attachment portion (a connecting portion) 200a of the base unit 200 is removably attached to a moving stage (receptor) 2a of the support stage 2. Even when the attachment portion 200a of the medical device 1 is removed from the moving stage 2a, the connection between the medical device 1 and the control device 3 is maintained so that the medical device 1 can be controlled by the control device 3. According to some embodiments, even when the attachment portion 200a of the medical device 1 is removed from the moving stage 2a, the medical device 1 and the support stage 2 are connected by the cable 5.

The user can manually move the medical device 1 with the medical device 1 removed from the support stage 2 (with the medical device 1 removed from the moving stage 2a) and insert the catheter 11 inside a subject.

The user can use the medical device 1 with the catheter 11 inserted into the subject and the medical device 1 attached to the support stage 2. More specifically, if the moving stage 2a moves with the medical device 1 attached to the moving stage 2a, the medical device 1 moves. Then, an operation to move the catheter 11 in a direction to insert it into the subject and an operation to move the catheter 11 in a direction to withdraw it from the subject are performed. The movement of the moving stage 2a is controlled by the control device 3.

In some embodiments, the attachment portion 200a of the base unit 200 includes a release switch and a removal switch (neither is illustrated). When the attachment portion 200a is attached to the moving stage 2a, the user can manually move the medical device 1 in the guiding direction of the moving stage 2a while holding down the release switch. That is, the moving stage 2a has a guide configuration to guide the movement of the medical device 1. When the user stops pressing the release switch, the medical device 1 is fixed to the moving stage 2a. If the removal switch is pressed with the attachment portion 200a mounted to the moving stage 2a, the user can remove the medical device 1 from the moving stage 2a.

A single switch may have the function of both a release switch and a removal switch. If the release switch has a mechanism that switches between the pressed state and unpressed state, the user need not continue to press the release switch during manual sliding movement of the medical device 1.

When the attachment portion 200a is attached to the moving stage 2a and the release switch and the removal switch are not pressed, the medical device 1 is fixed to the moving stage 2a and is moved by the moving stage 2a that is driven by a motor (not illustrated).

The medical device 1 includes a wire drive unit 300 (linear member drive unit, line drive unit, body drive unit) that drives the catheter 11. According to some embodiments, the medical device 1 is a robotic catheter device that drives the catheter 11 by a wire drive unit 300 controlled by the control device 3.

The control device 3 controls the wire drive unit 300 and can perform the operation of bending the catheter 11. According to some embodiments, the wire drive unit 300 is built into the base unit 200. More specifically, the base unit 200 includes a base housing 200f that houses the wire drive unit 300. That is, the base unit 200 includes the wire drive unit 300. The wire drive unit 300 and the base unit 200 are also collectively referred to as a catheter drive unit (base device, body).

The end where the front end of the catheter 11 inserted into the subject is disposed in the extension direction of the catheter 11 is referred to as a “distal end”. The end opposite to the distal end in the extension direction of the catheter 11 is referred to as a “proximal end”.

The catheter unit 100 includes a proximal-end cover 16 that covers the proximal end portion of the catheter 11. The proximal-end cover 16 has a tool hole 16a. A medical instrument can be inserted into the catheter 11 through the tool hole 16a.

As described above, according to some embodiments, the catheter 11 functions as a guiding device to guide the medical instrument to the desired position inside the subject.

For example, the catheter 11 is inserted to the target position inside the subject with an endoscope inserted into the catheter 11. At this time, at least one of the following is employed: a manual operation performed by the user, movement of the moving stage 2a, and driving of the catheter 11 by the wire drive unit 300. After the catheter 11 reaches the target position, the endoscope is withdrawn from the catheter 11 through the tool hole 16a. Then, a medical instrument is inserted through the tool hole 16a to collect various specimens from the inside of the subject and perform procedures inside of the subject or the like.

As described below, the catheter unit 100 is removably mounted to the catheter drive unit (base device, body) and, more specifically, to the base unit 200. After the medical device 1 has been used, the user can remove the catheter unit 100 from the base unit 200 and mount another catheter unit 100 to the base unit 200 to use the medical device 1 again.

As illustrated in FIG. 2, the medical device 1 includes an operating unit 400. According to some embodiments, the operating unit 400 is provided in the catheter unit 100. The operating unit 400 is operated by the user when the catheter unit 100 is fixed to the base unit 200 and when the catheter unit 100 is unfixed from the base unit 200.

By connecting the endoscope inserted into the catheter 11 to the monitor 4, an image captured by the endoscope can be displayed on the monitor 4. In addition, by connecting the monitor 4 to the control device 3, the monitor 4 can display the status of the medical device 1 and information related to the control of the medical device 1. For example, the position of the catheter 11 inside the subject and information related to the navigation of the catheter 11 inside the subject can be displayed on the monitor 4. The monitor 4, the control device 3, and the endoscope can be wired or wirelessly connected. The monitor 4 and the control device 3 may be connected via the support stage 2.

### Catheter

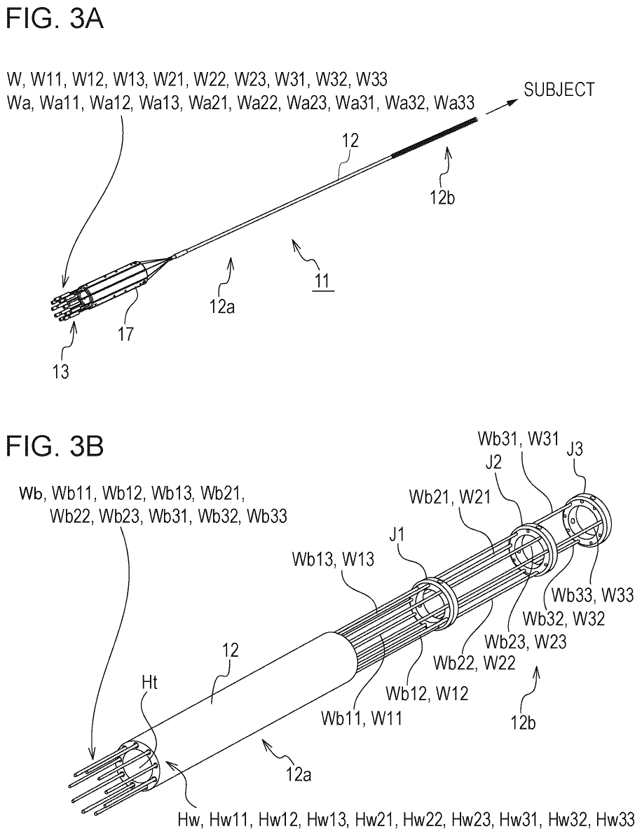

The catheter 11 serving as the bendable body is described below with reference to FIGS. 3A and 3B. FIGS. 3A and 3B illustrate the catheter 11. FIG. 3A illustrates the entire catheter 11. FIG. 3B is an enlarged view of the catheter 11.

The catheter 11 includes the bending section 12 (bending body, catheter body) 12 and a bending drive unit (catheter drive unit) 13 configured to bend the bending section 12. The bending drive unit 13 is configured to receive the driving force from the wire drive unit 300 via a joining device 21 (described below) and bend the bending section 12.

The catheter 11 is extended in the insertion direction of the catheter 11 into the subject. The extension direction (longitudinal direction) of the catheter 11 is the same as the extension direction (longitudinal direction) of the bending section 12 and the extension directions (longitudinal directions) of first to ninth drive wires (W11 to W33) (described below).

The bending drive unit 13 includes the plurality of drive wires (drive lines, linear members, linear actuators) connected to the bending section 12. More specifically, the bending drive unit 13 includes the first drive wire W11, the second drive wire W12, the third drive wire W13, the fourth drive wire W21, the fifth drive wire W22, the sixth drive wire W23, the seventh drive wire W31, the eighth drive wire W32, and the ninth drive wire W33.

Each of the first to ninth drive wires (W11 to W33) includes a retained portion (retained shaft, rod, or engaged portion) Wa. More specifically, the first drive wire W11 includes a first retained portion Wa11. The second drive wire W12 includes a second retained portion Wa12. The third drive wire W13 includes a third retained portion Wa13. The fourth drive wire W21 includes a fourth retained portion Wa21. The fifth drive wire W22 includes a fifth retained portion Wa22. The sixth drive wire W23 includes a sixth retained portion Wa23. The seventh drive wire W31 includes a seventh retained portion Wa31. The eighth drive wire W32 includes an eighth retained portion Wa32. The ninth drive wire W33 includes a ninth retained portion Wa33.

According to some embodiments, the first to ninth retained portions (Wa11 to Wa33) have the same shape.

Each of the first to ninth drive wires (W11 to W33) includes a flexible wire body (line body, linear body) Wb. More specifically, the first drive wire W11 includes a first wire body Wb11. The second drive wire W12 includes a second wire body Wb12. The third drive wire W13 includes a third wire body Wb13. The fourth drive wire W21 includes a fourth wire body Wb21. The fifth drive wire W22 includes a fifth wire body Wb22. The sixth drive wire W23 includes a sixth wire body Wb23. The seventh drive wire W31 includes a seventh wire body Wb31. The eighth drive wire W32 includes an eighth wire body Wb32. The ninth drive wire W33 includes a ninth wire body Wb33.

According to some embodiments, the first to third wire bodies (Wb11 to Wb13) have the same shape. The fourth to sixth wire bodies (Wb21 to Wb23) have the same shape. The seventh to ninth wire bodies (Wb31 to Wb33) have the same shape. According to some embodiments, the first to ninth wire bodies (Wb11 to Wb33) have the same shape except for their lengths.

The first to ninth retained portions (Wa11 to Wa33) are fixed to the first to ninth wire bodies (Wb11 to Wb33) at the proximal ends of the first to ninth wire bodies (Wb11 to Wb33), respectively.

The first to ninth drive wires (W11 to W33) are inserted into and fixed to the bending section 12 via the wire guide 17.

According to some embodiments, the material of each of the first to ninth drive wires (W11 to W33) is metal. However, the material of each of the first to ninth drive wires (W11 to W33) may be resin. The material of each of the first to ninth drive wires (W11 to W33) may include metal and resin.

Any one of the first to ninth drive wires (W11 to W33) is also referred to as a drive wire W. According to some embodiments, the first to ninth drive wires (W11 to W33) have the same shape except for the lengths of the first to ninth wire bodies (Wb11 to Wb33).

According to some embodiments, the bending section 12 is a tubular member that is flexible and has a passage Ht used to insert a medical instrument.

A wall surface of the bending section 12 has a plurality of wire holes used to allow the first to ninth drive wires (W11 to W33) to pass therethrough. More specifically, the wall surface of the bending section 12 has a first wire hole Hw11, a second wire hole Hw12, a third wire hole Hw13, a fourth wire hole Hw21, a fifth wire hole Hw22, a sixth wire hole Hw23, a seventh wire hole Hw31, an eighth wire hole Hw32, and a ninth wire hole Hw33. The first to ninth wire holes Hw (Hw11 to Hw33) correspond to the first to ninth drive wires (W11 to W33), respectively. In the reference numbers, the number following “Hw” indicates a corresponding drive wire number. For example, the first drive wire W11 is inserted into the first wire hole Hw11.

Any one of the first to ninth wire holes (Hw11 to Hw33) is also referred to as a wire hole Hw. According to some embodiments, the first to ninth wire holes (Hw11 to Hw33) have the same shape.

The bending section 12 has an intermediate region 12a and a bending region 12b. The bending region 12b is disposed at the distal end of the bending section 12. The bending region 12b has a first guide ring J1, a second guide ring J2, and a third guide ring J3 disposed therein. The bending region 12b is a region where the size and direction of bending of the bending section 12 can be controlled by the bending drive unit 13 that moves the first guide ring J1, the second guide ring J2, and the third guide ring J3. In FIG. 3B, part of the bending section 12 that covers the first to third guide rings (J1 to J3) is not illustrated.

According to some embodiments, the bending section 12 includes a plurality of auxiliary rings (not illustrated). In the bending region 12b, the first guide ring J1, the second guide ring J2, and the third guide ring J3 are fixed to the wall surface of the bending section 12. According to some embodiments, the plurality of auxiliary rings are disposed between the first guide ring J1 and the second guide ring J2 and between the second guide ring J2 and the third guide ring J3.

The medical instrument is guided to the front end of the catheter 11 by the passage Ht, the first to third guide rings (J1 to J3), and the plurality of auxiliary rings.

Each of the first to ninth drive wires (W11 to W33) passes through the intermediate region 12a and is fixed to one of the first to third guide rings (J1 to J3).

More specifically, the first drive wire W11, second drive wire W12, and third drive wire W13 are fixed to the first guide ring J1. The fourth drive wire W21, fifth drive wire W22, and sixth drive wire W23 are fixed to the second guide ring J2 through the first guide ring J1 and the plurality of auxiliary rings. The seventh drive wire W31, eighth drive wire W32, and ninth drive wire W33 are fixed to the third guide ring J3 through the first guide ring J1, the second guide ring J2, and the plurality of auxiliary rings.

The medical device 1 can bend the bending section 12 toward a direction that intersects the extension direction of the catheter 11 by driving the wire drive unit 300, and thus, driving the bending drive unit 13. More specifically, by moving each of the first to ninth drive wires (W11 to W33) in the extension direction of the bending section 12, the bending region 12b of the bending section 12 can be bent in a direction that intersects the extension direction via the first to third guide rings (J1 to J3). That is, the first to ninth drive wires (W11 to W33) can bend the bending region 12b of the bending section 12 by moving the first to ninth drive wires (W11 to W33) in their respective extension directions.

The user can insert the catheter 11 to the desired part inside the subject by using at least one of the following methods: moving the medical device 1 manually or by using the moving stage 2a and bending the bending section 12.

According to some embodiments, the first to third guide rings (J1 to J3) are moved by the first to ninth drive wires (W11 to W33) to bend the bending section 12. However, the present invention is not limited to the configuration. Any one or two of the first to third guide rings (J1 to J3) and the drive wires fixed to the guide rings may be removed.

For example, the catheter 11 may have a configuration with the seventh to ninth drive wires (W31 to W33) and the third guide ring J3 and without the first to sixth drive wires (W11 to W23) and the first to second guide rings (J1 to J2). Alternatively, the catheter 11 may have a configuration with the fourth to ninth drive wires (W21 to W33) and the second to third guide rings (J2 to J3) and without the first to third drive wires (W11 to W13) and the first guide ring J1.

The catheter 11 may have a configuration to drive one guide ring by using two drive wires. Even in the configuration, the number of guide rings may be one or more than one.

### Catheter Unit

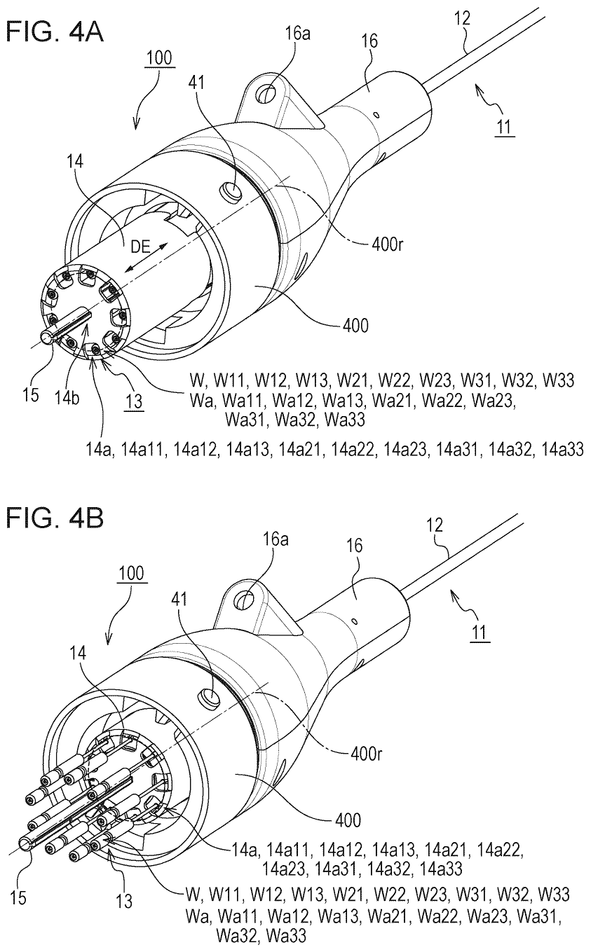

The catheter unit 100 is described below with reference to FIGS. 4A and 4B.

FIGS. 4A and 4B illustrate the catheter unit 100. FIG. 4A illustrates the catheter unit 100 with a wire cover 14 (described below) in a covering position. FIG. 4B illustrates the catheter unit 100 with the wire cover 14 (described below) in an exposure position.

The catheter unit 100 includes a catheter 11 including the bending section 12 and the bending drive unit 13, and a proximal-end cover 16 that supports the proximal end of the catheter 11. The catheter unit 100 includes a cover (wire cover) 14 for covering and protecting the first to ninth drive wires (W11 to W33), which are the plurality of drive wires.

The catheter unit 100 is mountable and dismountable to and from the base unit 200 in a mounting/dismounting direction DE. The mounting direction of the catheter unit 100 to the base unit 200 and the dismounting direction of the catheter unit 100 from the base unit 200 are parallel to the mounting/dismounting direction DE.

The proximal end cover (frame, bending section housing, catheter housing) 16 is a cover that covers part of the catheter 11. The proximal-end cover 16 has the tool hole 16a used to insert a medical instrument into the passage Ht of the bending section 12.

The wire cover 14 has a plurality of exposure holes (wire cover holes, cover holes) that allow the first to ninth drive wires (W11 to W33) to pass therethrough. The wire cover 14 has a first exposure hole 14a11, a second exposure hole 14a12, a third exposure hole 14a13, a fourth exposure hole 14a21, a fifth exposure hole 14a22, a sixth exposure hole 14a23, a seventh exposure hole 14a31, an eighth exposure hole 14a32, and a ninth exposure hole 14a33. The first to ninth exposure holes (14a11 to 14a33) correspond to the first to ninth drive wires (W11 to W33), respectively. In the reference numbers, the number following “14a” indicates the corresponding drive wire number. For example, the first drive wire W11 is inserted into the first exposure hole 14a11.

Any one of the first to ninth exposure holes (14a11 to 14a33) is also referred to as an exposure hole 14a. According to some embodiments, the first to ninth exposure holes (14a11 to 14a33) have the same shape.

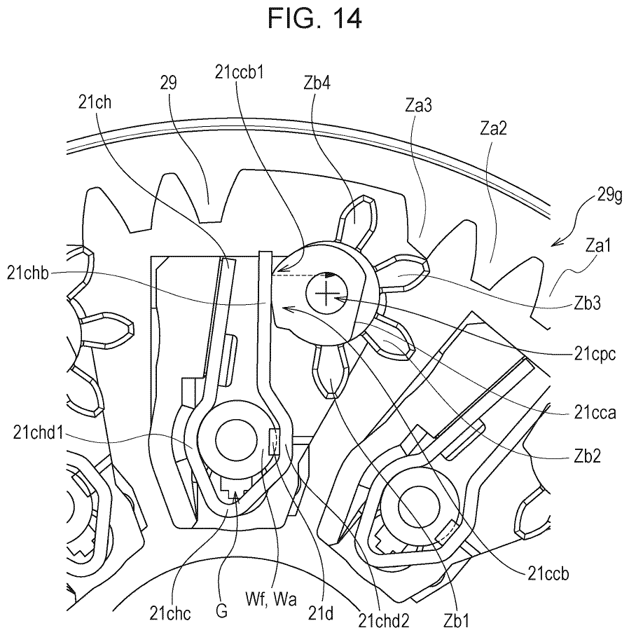

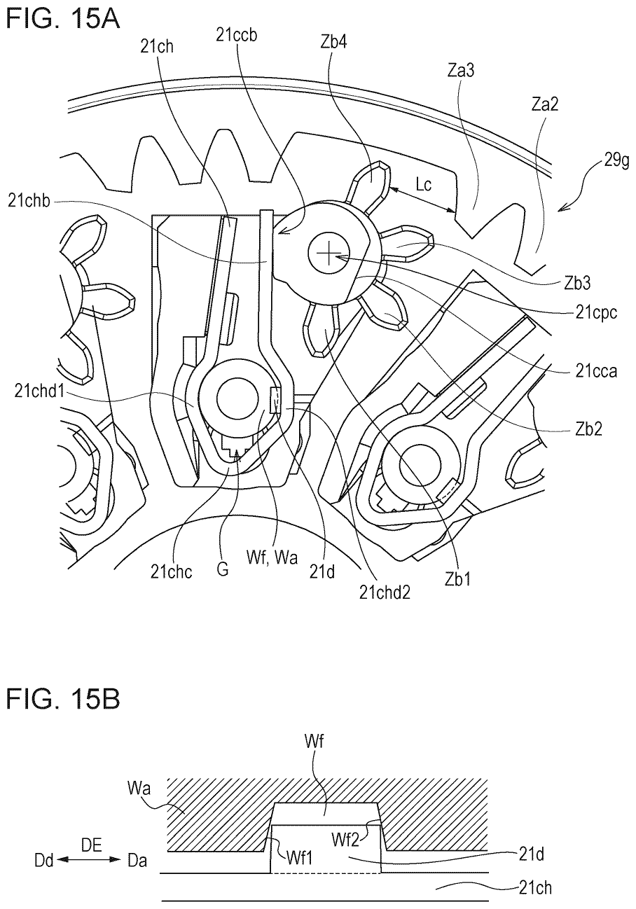

The wire cover 14 can be moved to a covering position (refer to FIG. 15A), where the wire cover 14 covers the first to ninth drive wires (W11 to W33), or a retracted non-covering position (refer to FIG. 15B), where the wire cover 14 is retracted from the covering position. The retracted non-covering position is also referred to as an “exposure position” where the first to ninth drive wires (W11 to W33) are exposed.

Before the catheter unit 100 is mounted to the base unit 200, the wire cover 14 is located at the covering position. When the catheter unit 100 is mounted to the base unit 200, the wire cover 14 moves from the covering position to the exposure position in the mounting/dismounting direction DE.

According to some embodiments, after being moved from the covering position to the exposure position, the wire cover 14 remains in the exposure position. Therefore, after the catheter unit 100 is mounted to the base unit 200, the wire cover 14 remains in the exposure position even if the catheter unit 100 is dismounted from the base unit 200.

However, the wire cover 14 may be configured to return to the covering position after being moved from the covering position to the exposure position. For example, the catheter unit 100 may include a biasing member that biases the wire cover 14 from the exposure position toward the covering position. In this case, if, after the catheter unit 100 is mounted to the base unit 200, the catheter unit 100 is dismounted from the base unit 200, the wire cover 14 is moved from the exposure position to the covering position.

When the wire cover 14 is located at the exposure position, the first to ninth retained portions (Wa11 to Wa33) of the first to ninth drive wires (W11 to W33) are exposed. As a result, joining between the bending drive unit 13 and the joining device 21 (described below) is allowed. When the wire cover 14 is located at the exposure position, the first to ninth retained portions (Wa11 to Wa33) of the first to ninth drive wires (W11 to W33) protrude from the first to ninth exposure holes (14a11 to 14a33), respectively. More specifically, the first to ninth retained portions (Wa11 to Wa33) protrude from the first to ninth exposure holes (14a11 to 14a33) in a mounting direction Da (described below).

As illustrated in FIG. 4B, the first to ninth drive wires (W11 to W33) are arranged along a circle (imaginary circle) with a predetermined radius.

According to some embodiments, the catheter unit 100 includes a key shaft (key, catheter-side key) 15. According to some embodiments, the key shaft 15 extends in the mounting/dismounting direction DE. The wire cover 14 has a shaft hole 14b that allows the key shaft 15 to pass therethrough. The key shaft 15 can be engaged with a key receiving portion 22 (described below). When the key shaft 15 is engaged with the key receiving portion 22, the movement of the catheter unit 100 relative to the base unit 200 in the circumference direction of the circle (imaginary circle) along which the first to ninth drive wires (W11 to W33) are arranged is restricted so as to be within a predetermined range.

According to some embodiments, the first to ninth drive wires (W11 to W33) are arranged on the outer side of the key shaft 15 so as to surround the key shaft 15 as viewed in the mounting/dismounting direction DE. That is, the key shaft 15 is disposed on the inner side of the circle (imaginary circle) along which the first to ninth drive wires (W11 to W33) are arranged. Therefore, the key shaft 15 and the first to ninth drive wires (W11 to W33) can be disposed in a space-saving manner.

According to some embodiments, the catheter unit 100 includes the operating unit 400. The operating unit 400 is movable (rotatable) relative to the proximal-end cover 16 and the bending drive unit 13. The operating unit 400 is rotatable about a rotation axis 400r. The rotation axis 400r of the operating unit 400 extends in the mounting/dismounting direction DE.

The operating unit 400 is configured to be movable (rotatable) relative to the base unit 200 with the catheter unit 100 mounted to the base unit 200. More specifically, the operating unit 400 is configured to be movable (rotatable) relative to the base housing 200f, the wire drive unit 300, and the joining device 21 (described below).

### Base Unit

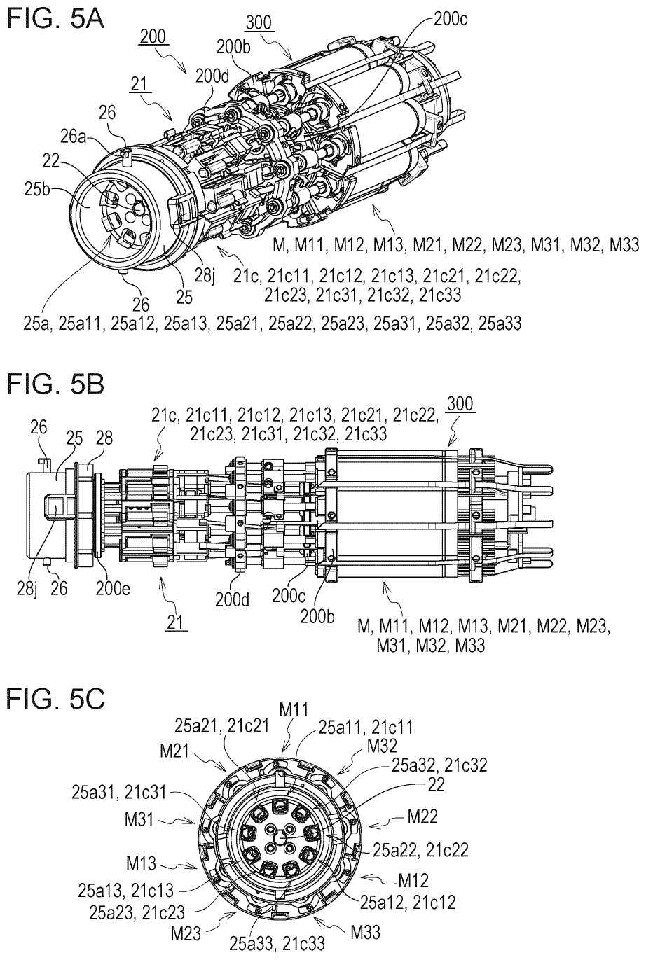

The base unit 200 and the wire drive unit 300 are described below with reference to FIGS. 5A to 5C.

FIGS. 5A to 5C illustrate the base unit 200 and the wire drive unit 300. FIG. 5A is a perspective view of the internal configuration of the base unit 200. FIG. 5B is a side view of the internal configuration of the base unit 200. FIG. 5C is a view of the base unit 200 as viewed in the mounting/dismounting direction DE.

As described above, the medical device 1 includes the base unit 200 and the wire drive unit 300. According to some embodiments, the wire drive unit 300 is housed in the base housing 200f and is provided inside of the base unit 200. That is, the base unit 200 includes the wire drive unit 300.

The wire drive unit 300 includes a plurality of drive sources (motors). According to some embodiments, the wire drive unit 300 includes a first drive source M11, a second drive source M12, a third drive source M13, a fourth drive source M21, a fifth drive source M22, a sixth drive source M23, a seventh drive source M31, an eighth drive source M32, and a ninth drive source M33.

Any one of the first to ninth drive sources (M11 to M33) is referred to as a drive source M. According to some embodiments, the first to ninth drive sources (M11 to M33) have the same configuration.

The base unit 200 includes the joining device 21. The joining device 21 is housed in the base housing 200f. The joining device 21 is connected to the wire drive unit 300. The joining device 21 includes a plurality of joining sections. According to some embodiments, the joining device 21 includes a first joining section 21c11, a second joining section 21c12, a third joining section 21c13, a fourth joining section 21c21, a fifth joining section 21c22, a sixth joining section 21c23, a seventh joining section 21c31, an eighth joining section 21c32, and a ninth joining section 21c33.

Any one of the first to ninth joining sections (21c11 to 21c33) is also referred to as a joining section 21c. According to some embodiments, the first to ninth joining sections (21c11 to 21c33) have the same configuration.

Each of the plurality of joining sections is connected to one of the plurality of drive sources and is driven by the drive source. More specifically, the first joining section 21c11 is connected to the first drive source M11 and is driven by the first drive source M11. The second joining section 21c12 is connected to the second drive source M12 and is driven by the second drive source M12. The third joining section 21c13 is connected to the third drive source M13 and is driven by the third drive source M13. The fourth joining section 21c21 is connected to the fourth drive source M21 and is driven by the fourth drive source M21. The fifth joining section 21c22 is connected to the fifth drive source M22 and is driven by the fifth drive source M22. The sixth joining section 21c23 is connected to the sixth drive source M23 and is driven by the sixth drive source M23. The seventh joining section 21c31 is connected to the seventh drive source M31 and is driven by the seventh drive source M31. The eighth joining section 21c32 is connected to the eighth drive source M32 and is driven by the eighth drive source M32. The ninth joining section 21c33 is connected to the ninth drive source M33 and is driven by the ninth drive source M33.

As described below, the bending drive unit 13 including the first to ninth drive wires (W11 to W33) is connected to the joining device 21. The bending drive unit 13 receives the driving force of the wire drive unit 300 via the joining device 21 and bends the bending section 12.

The drive wire W is joined to the joining section 21c via the retained portion Wa. Each of the plurality of drive wires is joined to one of the plurality of joining sections.

More specifically, the first retained portion Wa11 of the first drive wire W11 is joined to the first joining section 21c11. The second retained portion Wa12 of the second drive wire W12 is joined to the second joining section 21c12. The third retained portion Wa13 of the third drive wire W13 is joined to the third joining section 21c13. The fourth retained portion Wa21 of the fourth drive wire W21 is joined to the fourth joining section 21c21. The fifth retained portion Wa22 of the fifth drive wire W22 is joined to the fifth joining section 21c22. The sixth retained portion Wa23 of the sixth drive wire W23 is joined to the sixth joining section 21c23. The seventh retained portion Wa31 of the seventh drive wire W31 is joined to the seventh joining section 21c31. The eighth retained portion Wa32 of the eighth drive wire W32 is joined to the eighth joining section 21c32. The ninth retained portion Wa33 of the ninth drive wire W33 is joined to the ninth joining section 21c33.

The base unit 200 includes a base frame 25. The base frame 25 has a plurality of insertion holes that allow the first to ninth drive wires (W11 to W33) to pass therethrough. The base frame 25 has a first insertion hole 25a11, a second insertion hole 25a12, third insertion holes 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes (25a11 to 25a33) correspond to the first to ninth drive wires (W11 to W33), respectively. In the reference numbers, the number following “25a” indicates a corresponding drive wire number. For example, the first drive wire W11 is inserted into the first insertion hole 25a11.

Any one of the first to ninth insertion holes (25a11 to 25a33) is also referred to as an insertion hole 25a. According to some embodiments, the first to ninth insertion holes (25a11 to 25a33) have the same shape.

The base frame 25 has a mounting opening 25b into which the wire cover 14 is inserted. The first to ninth insertion holes (25a11 to 25a33) are disposed at the bottom of the mounting opening 25b.

In addition, the base unit 200 includes a motor frame 200b, a first bearing frame 200c, a second bearing frame 200d, and a third bearing frame 200e. The motor frame 200b, the first bearing frame 200c, the second bearing frame 200d, and the third bearing frame 200e are joined.

The base frame 25 includes the key receiving portion 22 (keyhole, base-side key, body-side key) that receives the key shaft 15. The engagement of the key shaft 15 with the key receiving portion 22 prevents the catheter unit 100 from being mounted to the base unit 200 in a wrong phase.

The engagement of the key shaft 15 with the key receiving portion 22 restricts the movement of the catheter unit 100 relative to the base unit 200 in the circumference direction of the circle (imaginary circle) in which the first to ninth drive wires (W11 to W33) are arranged so that the movement is within a predetermined range.

As a result, the first to ninth drive wires (W11 to W33) are engaged with the first to ninth insertion holes (25a11 to 25a33) and the first to ninth joining sections (21c11 to 21c33), respectively. That is, the drive wire W is prevented from being engaged with a different insertion hole 25a than the corresponding insertion hole 25a and a different joining section 21c than the corresponding joining section 21c.

The user can correctly join the first to ninth drive wires (W11 to W33) to the first to ninth joining sections (21c11 to 21c33), respectively, by engaging the key shaft 15 with the key receiving portion 22. Thus, the user can easily mount the catheter unit 100 to the base unit 200.

According to some embodiments, the key shaft 15 has a protrusion protruding in a direction that intersects the mounting/dismounting direction DE, and the key receiving portion 22 has a recess into which the protrusion is inserted. In the circumferential direction, the position where the protrusion and the recess are engaged is the position where the drive wire W is engaged with the corresponding insertion hole 25a and the corresponding joining section 21c.

The key shaft 15 can be disposed on one of the base unit 200 and the catheter unit 100, and the key receiving portion 22 can be disposed on the other. For example, the key shaft 15 may be disposed on the side adjacent to the base unit 200, and the key receiving portion 22 may be disposed on the side adjacent to the catheter unit 100.

The base unit 200 includes a joint 28 having a joint engagement portion 28j. The base frame 25 includes a locking shaft 26 having a locking protrusion 26a. The functions thereof are described below.

### Joining of Motor and Drive Wire

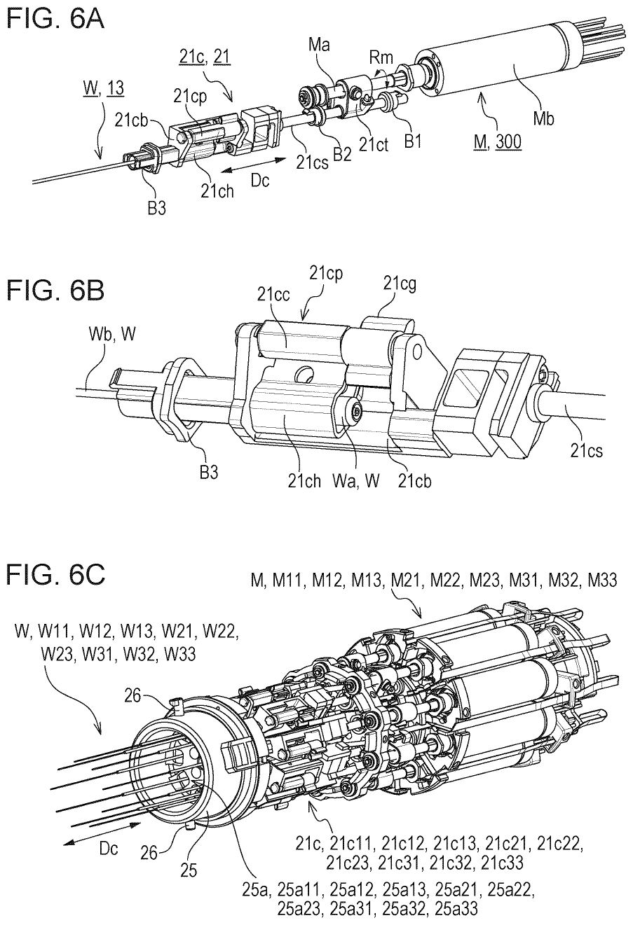

The joining of the wire drive unit 300, the joining device 21, and the bending drive unit 13 are described below with reference to FIGS. 6A, 6B, and 6C.

FIGS. 6A, 6B, and 6C illustrate the wire drive unit 300, the joining device 21, and the bending drive unit 13. FIG. 6A is a perspective view of the drive source M, the joining section 21c, and the drive wire W. FIG. 6B is an enlarged view of the joining section 21c and the drive wire W. FIG. 6C is a perspective view illustrating the joining of the wire drive unit 300, the joining device 21, and the bending drive unit 13.

According to some embodiments, the configurations in which the first to ninth drive wires (W11 to W33) are joined to the first to ninth joining sections (21c11 to 21c33), respectively, are the same. In addition, the configurations in which the first to ninth joining sections (21c11 to 21c33) are joined to the first to ninth drive sources (M11 to M33), respectively, are the same. Therefore, the connection configuration is described below with reference to one drive wire W, one joining section 21c, and one drive source M.

As illustrated in FIG. 6A, the drive source M includes an output shaft Ma and a motor body Mb that rotates the output shaft Ma in a rotation direction Rm. The surface of the output shaft Ma has a spiral groove. The output shaft Ma has a so-called screw shape. The motor body Mb is fixed to the motor frame 200b.

The joining section 21c includes a tractor 21ct connected to the output shaft Ma and a tractor support shaft 21cs that supports the tractor 21ct. The tractor support shaft 21cs is connected to a connecting base 21cb.

The joining section 21c includes a leaf spring 21ch serving as a retaining portion configured to retain the retained portion Wa of the drive wire W. The drive wire W is engaged with the joining section 21c through the insertion hole 25a. More specifically, the retained portion Wa is engaged with the leaf spring 21ch. As described below, the leaf spring 21ch can have two states, a state in which the retained portion Wa is fixed (a fixed state), and a state in which the retained portion Wa is released (a released state).

The joining section 21c includes a pressure member (first rotating body) 21cp. The pressure member 21cp includes a gear section 21cg that meshes with an internal gear 29 (described below) and a cam 21cc serving as a pressure portion for pressing the leaf spring 21ch.

As described below, the cam 21cc can move relative to the leaf spring 21ch. By moving the cam 21cc, the leaf spring 21ch is switched between the fixed state and the released state.

The joining section 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. Therefore, when the motor shaft Ma rotates in the rotation direction Rm, the joining section 21c is restricted from rotating about the motor shaft Ma. The first bearing B1, the second bearing B2, and the third bearing B3 are provided for each of the first to ninth joining sections (21c11 to 21c33).

Since the rotation of the joining section 21c about the motor shaft Ma is regulated, a force in the rotation axis direction of the motor shaft Ma is exerted on the tractor 21ct by the spiral groove of the motor shaft Ma when the motor shaft Ma rotates. As a result, the joining section 21c moves in the rotation axis direction of the motor shaft Ma (a direction Dc). The movement of the joining section 21c moves the drive wire W and, thus, bends the bending section 12.

That is, the motor shaft Ma and the tractor 21ct constitute a so-called feed screw that converts the rotational motion transmitted from the drive source M into linear motion by using a screw. According to some embodiments, the motor shaft Ma and the tractor 21ct form a slide screw, but a ball screw may be used.

As illustrated in FIG. 6C, by mounting the catheter unit 100 to the base unit 200, the first to ninth drive wires (W11 to W33) are joined to the first to ninth joining sections (21c11 to 21c33), respectively.

The control device 3 can control each of the first to ninth drive sources (M11 to M33) independently. That is, any drive source among the first to ninth drive sources (M11 to M33) can operate or stop independently regardless of whether the other drive sources are stopped or not. In other words, the control device 3 can control the first to ninth drive wires (W11 to W33) independently of each other. As a result, the first to third guide rings (J1 to J3) can be controlled independently of each other, and the bending region 12b of the bending section 12 can bend in any direction.

### Mounting of Catheter Unit

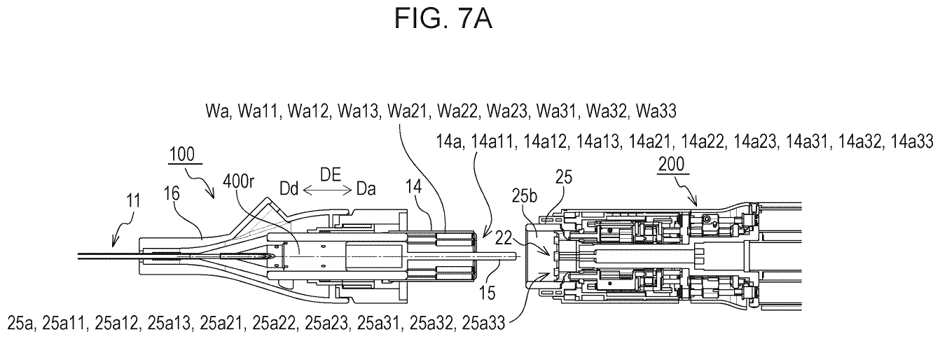

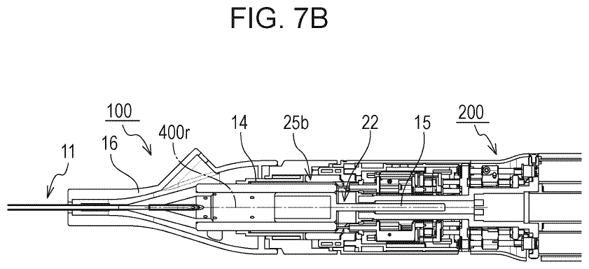

The mounting operation of the catheter unit 100 to the base unit 200 is described below with reference to FIGS. 7A and 7B.

FIGS. 7A and 7B illustrate the mounting of the catheter unit 100. FIG. 7A illustrates the catheter unit 100 before the catheter unit 100 is mounted to the base unit 200. FIG. 7B illustrates the catheter unit 100 after the catheter unit 100 is mounted to the base unit 200.

According to some embodiments, the mounting/dismounting direction DE of the catheter unit 100 is the same as the direction of the rotation axis 400r of the operating unit 400. Of the mounting/dismounting direction DE, the direction in which the catheter unit 100 is mounted to the base unit 200 is referred to as a mounting direction Da. Of the mounting/dismounting direction DE, the direction in which the catheter unit 100 is dismounted from the base unit 200 (the opposite direction of the mounting direction Da) is referred to as a dismounting direction Dd.

As illustrated in FIG. 7A, before the catheter unit 100 is mounted to the base unit 200, the wire cover 14 is in the covering position. At this time, the wire cover 14 covers the first to ninth drive wires (W11 to W33) so that the first to ninth retained portions (Wa11 to Wa33) do not protrude from the first to ninth exposure holes (14a11 to 14a33) of the wire cover 14. Therefore, the first to ninth drive wires (W11 to W33) can be protected before the catheter unit 100 is mounted to the base unit 200.

When the catheter unit 100 is mounted to the base unit 200, the key shaft 15 is engaged with the key receiving portion 22. The key shaft 15 protrudes from the wire cover 14. According to some embodiments, the wire cover 14 is not engaged with the mounting opening 25b when the key shaft 15 reaches the entrance of the key receiving portion 22. That is, when the phase of the catheter unit 100 relative to the base unit 200 is a phase where the key shaft 15 and the key receiving portion 22 cannot be engaged with each other, the wire cover 14 is not engaged with the mounting opening 25b and remains in the covering position. Therefore, even when the catheter unit 100 is moved so that the key shaft 15 and the key receiving portion 22 are engaged with each other, the first to ninth drive wires (W11 to W33) are protected.

When the key shaft 15 and the key receiving portion 22 are engaged and, then, the catheter unit 100 is moved in the mounting direction Da relative to the base unit 200, the catheter unit 100 is mounted on the base unit 200. By mounting the catheter unit 100 to the base unit 200, the wire cover 14 moves to the exposure position. According to some embodiments, the wire cover 14 moves from the covering position to the exposure position by being brought into contact with the base frame 25 (refer to FIG. 7B).

More specifically, when the catheter unit 100 is mounted, the wire cover 14 is brought into contact with the base frame 25 and, thus, stops. At this time, by moving the catheter unit 100 in the mounting direction Da, the wire cover 14 moves relative to the portion of the catheter unit 100 other than the wire cover 14. As a result, the wire cover 14 moves from the covering position to the exposure position.

While the wire cover 14 moves from the covering position to the exposure position, the retained portion Wa of the drive wire W protrudes from the exposure hole 14a of the wire cover 14 and is inserted into the insertion hole 25a. Then, the retained portion Wa is engaged with the leaf spring 21ch of the joining section 21c (refer to FIG. 6B).

When the catheter unit 100 is simply mounted to the base unit 200, the catheter unit 100 can be dismounted by moving the catheter unit 100 relative to the base unit 200 in the dismounting direction Dd. In addition, as described below, when the catheter unit 100 is simply mounted to the base unit 200, the drive wire W and the joining section 21c are not fixed.

By operating the operating unit 400 with the catheter unit 100 mounted to the base unit 200, the catheter unit 100 is prevented from being dismounted from the base unit 200. Furthermore, by operating the operating unit 400 with the catheter unit 100 mounted to the base unit 200, the bending drive unit 13 is fixed to the joining device 21, and the bending drive unit 13 is joined to the wire drive unit 300 via the joining device 21.

### Fixing and Unfixing of Bending Drive Unit

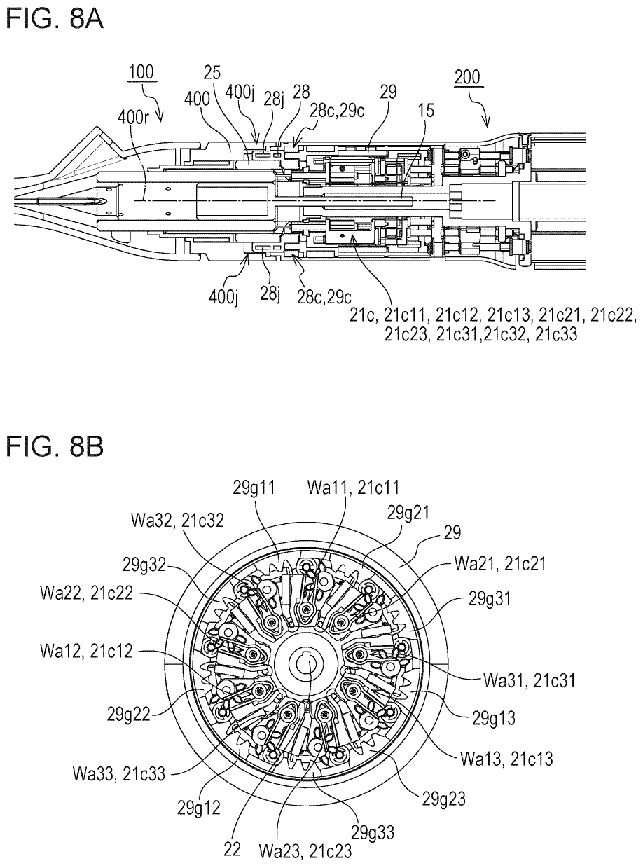

A configuration for fixing the bending drive unit 13 to the joining device 21 and a configuration for unfixing the bending drive unit 13 by the joining device 21 are described below with reference to FIGS. 8A, 8B, 9, 10A to 10E, 11A, 11B, 12, 13, 14, 15A, and 15B.

FIGS. 8A and 8B illustrate the joining of catheter unit 100 and the base unit 200. FIG. 8A is a cross-sectional view of the catheter unit 100 and the base unit 200. FIG. 8A is a cross-sectional view of the catheter unit 100 and the base unit 200 cut along the rotation axis 400r. FIG. 8B is a cross-sectional view of the base unit 200. FIG. 8B is a cross-sectional view of the base unit 200 cut at the position of the joining section 21c in a direction perpendicular to the rotation axis 400r.

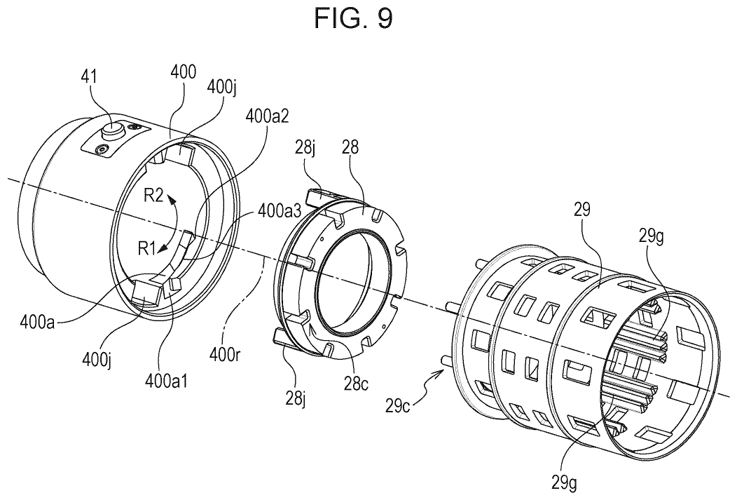

FIG. 9 is an exploded view illustrating the joining of the catheter unit 100 and the base unit 200.

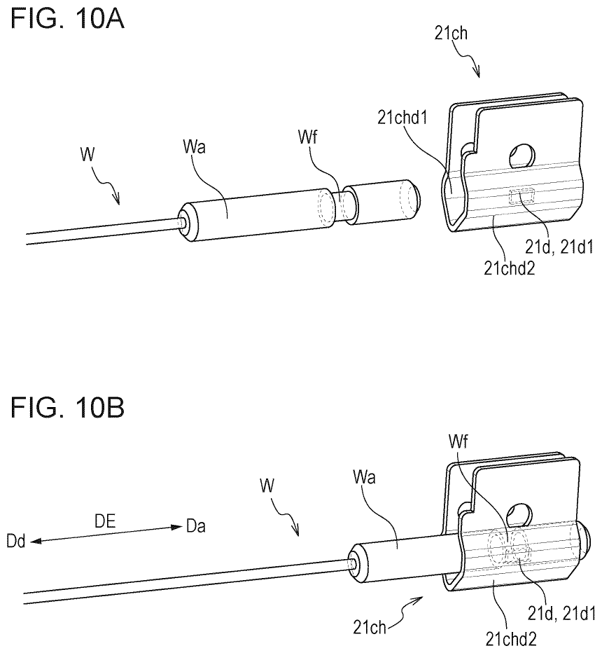

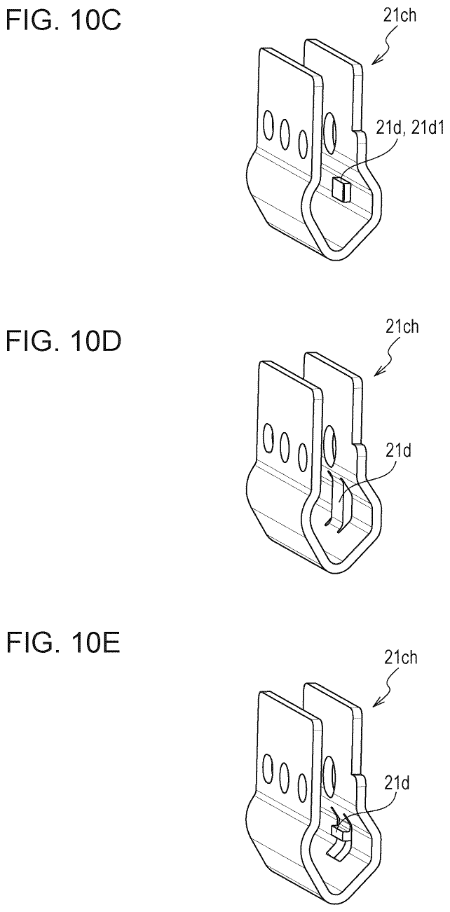

FIGS. 10A to 10E illustrate the retention of the retained portion Wa by the leaf spring 21ch according to some embodiments. FIG. 10A illustrates the leaf spring 21ch and the retained portion Wa that are separated from each other, and FIG. 10B illustrates the leaf spring 21ch and the retained portion Wa that are engaged with each other. FIG. 10C illustrates a protrusion 21d formed by a protrusion forming member 21dl fixed to the leaf spring 21ch. FIGS. 10D and 10E illustrate the protrusion 21d formed by a bent portion of the leaf spring 21ch.

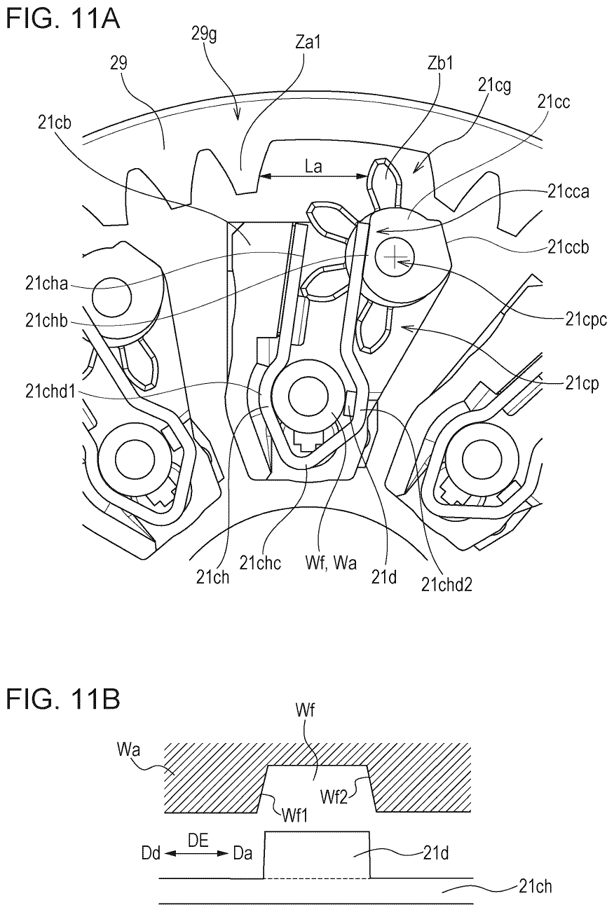

FIGS. 11A, 11B, 12, 13, 14, 15A, and 15B illustrate the fixing of the drive wire W by the joining section 21c.

As illustrated in FIGS. 8A and 9, the base unit 200 includes the joint (intermediate member, second transmission member) 28 and the internal gear 29 serving as a moving gear (interlocking gear, transmission member, first transmission member) that is linked with the operating unit 400 via the joint 28.

The joint 28 includes a plurality of transmitting portions 28c, and the internal gear 29 includes a plurality of transmitted portions 29c. The plurality of transmitting portions 28c are engaged with the plurality of transmitted portions 29c. When the joint 28 rotates, the rotation of the joint 28 is transmitted to the internal gear 29.

When the catheter unit 100 is mounted to the base unit 200, an engagement portion 400j provided in the operating unit 400 is engaged with the joint engagement portion 28j of the joint 28. When the operating unit 400 rotates, the rotation of the operating unit 400 is transmitted to the joint 28. The operating unit 400, the joint 28, and the internal gear 29 rotate in the same direction.

The internal gear 29 includes a plurality of tooth portions used to switch the state in which the first to ninth joining sections (21c11 to 21c33) fix the first to ninth drive wires (W11 to W33), respectively, and the state in which the first to ninth joining sections (21c11 to 21c33) unfix the first to ninth drive wires (W11 to W33), respectively. Each of the plurality of tooth portions (action portions, switching gear portions) of the internal gear 29 is engaged with the gear section 21cg of the pressure member 21cp of one of the first to ninth joining sections (21cl1 to 21c33).

More specifically, according to some embodiments, the internal gear 29 includes a first tooth portion 29g11, a second tooth portion 29g12, a third tooth portion 29g13, a fourth tooth portion 29g21, a fifth tooth portion 29g22, a sixth tooth portion 29g23, a seventh tooth portion 29g31, an eighth tooth portion 29g32, and a ninth tooth portion 29g33. The first to ninth tooth portions (29g11 to 29g33) are formed with a gap between each other.

The first tooth portion 29g11 meshes with the gear section 21cg of the first joining section 21cl1. The second tooth portion 29g12 meshes with the gear section 21cg of the second joining section 21c12. The third tooth portion 29g13 meshes with the gear section 21cg of the third joining section 21c13. The fourth tooth portion 29g21 meshes with the gear section 21cg of the fourth joining section 21c21. The fifth tooth portion 29g22 meshes with the gear section 21cg of the fifth joining section 21c22. The sixth tooth portion 29g23 meshes with the gear section 21cg of the sixth joining section 21c23. The seventh tooth portion 29g31 meshes with the gear section 21cg of the seventh joining section 21c31. The eighth tooth portion 29g32 meshes with the gear section 21cg of the eighth joining section 21c32. The ninth tooth portion 29g33 meshes with the gear section 21cg of the ninth joining section 21c33.

Any one of the first to ninth tooth portions (29g11 to 29g33) is also referred to as a tooth portion 29g. According to some embodiments, the first to ninth tooth portions (29g11 to 29g33) have the same configuration.

According to some embodiments, the configurations in which the first to ninth drive wires (W11 to W33) are connected to the first to ninth joining sections (21c11 to 21c33), respectively, are the same. In addition, the configurations in which the first to ninth joining sections (21c11 to 21c33) are connected to the first to ninth tooth portions (29g11 to 29g33), respectively, are the same. Therefore, the connection configuration is described below with reference to one drive wire W, one joining section 21c, and one tooth portion 29g.

In each of the first to ninth joining sections (21c11 to 21c33), the gear section 21cg is moved by the internal gear 29 and, thus, the pressure member 21cp rotates. The pressure member 21cp can rotate to a pressing position in which the cam 21cc presses the leaf spring 21ch and to a retracted position in which the cam 21cc is retracted from the pressing position.

Rotation of the operating unit 400 causes the internal gear 29 to rotate. Rotation of the internal gear 29 causes each of the first to ninth joining sections (21c11 to 21c33) to operate. That is, the motion of rotating the single operating unit 400 can cause the first to ninth joining sections (21c11 to 21c33) to operate.

The operating unit 400 can move to the fixation position (locked position, first position) and to the dismount position (second position, mount/dismount position) with the catheter unit 100 mounted to the base unit 200. As described below, the operating unit 400 can also be moved to a release position (third position) with the catheter unit 100 mounted to the base unit 200. The release position is located between the fixation position and the dismount position in the circumferential direction of the operating unit 400. As described below, when the operating unit 400 is in the dismount position, the catheter unit 100 is allowed to be mounted to and dismounted from the base unit 200. When the operating unit 400 is in the fixation position or release position, mounting and dismounting of the catheter unit 100 to and from the base unit 200 is restricted.

When the catheter unit 100 is mounted to the base unit 200, the operating unit 400 is in the dismount position. When the catheter unit 100 is mounted to the base unit 200 and the operating unit 400 is in the dismount position, the fixation (lock) of the drive wire W to the joining section 21c is released. This state is referred to as an unlocked state of the joining section 21c. The state in which the drive wire W is fixed (locked) to the joining section 21c is referred to as a locked state of the joining section 21c.

That is, the operating unit 400, the joint 28, and the internal gear 29 function as a second rotating body that moves the pressure member 21cp from the pressing position to the retracted position.

The operation performed when the drive wire W is fixed to the joining section 21c is described below with reference to FIGS. 10A to 10E, 11A, 11B, 12, 13, 14, 15A, and 15B.

As illustrated in FIGS. 10A and 10B, a second portion 21chd2 of the leaf spring 21ch has the protrusion 21d serving as an insertion portion. The retained portion Wa has a recess Wf serving as an inserted portion. The recess Wf is provided over the entire circumference (360°) of the retained portion Wa having a cylindrical shape in the circumferential direction. Therefore, if the recess Wf and the protrusion 21d are at the same position in the axial direction of the retained portion Wa, the recess Wf and the protrusion 21d can be engaged.

According to some embodiments, as illustrated in FIG. 10C, the protrusion 21d is formed by the protrusion forming member 21dl integrated with the leaf spring 21ch. The protrusion forming member 21dl that forms the protrusion 21d is fixed to the leaf spring 21ch. By using adhesion, welding, a double-stick tape, or the like, the protrusion forming member 21dl can be fixed to the leaf spring 21ch. In this configuration, the protrusion 21d (the protrusion forming member 21d1) moves integrally with the leaf spring 21ch. The protrusion forming member 21dl and the leaf spring 21ch are also referred to as part of the elastic member that holds the retained portion Wa. That is, the elastic member that retains the retained portion Wa of the drive wire W includes the protrusion forming member 21dl (the protrusion 21d) and the leaf spring 21ch.

As illustrated in FIGS. 10D and 10E, the protrusion 21d may be formed by bending part of the leaf spring 21ch.

By mounting the catheter unit 100 to the base unit 200, the protrusion 21d and the recess Wf overlap in the mounting/dismounting direction DE.

After the catheter unit 100 is mounted to the base unit 200 and before the operating unit 400 is operated, the catheter unit 100 can be dismounted from the base unit 200. The state in which the catheter unit 100 can be dismounted from the base unit 200 is hereinafter referred to as a dismountable state.

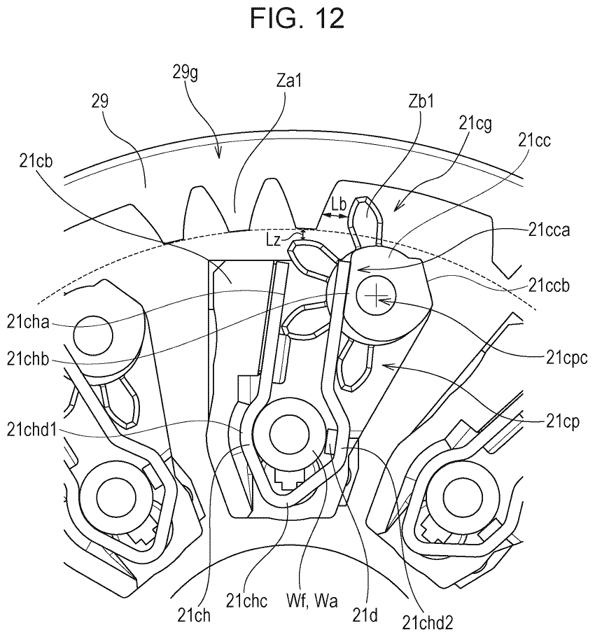

FIG. 11A illustrates the internal gear 29 and joining section 21c in the dismountable state. FIG. 11A illustrates the internal gear 29 and the joining section 21c when the operating unit 400 is located at the dismount position. FIG. 11A is a view of the joining section 21c as viewed from the mounting/dismounting direction DE. That is, FIG. 11A is a view of the joining section 21c as viewed from the extension direction of the drive wire W. The same applies to FIGS. 12 to 15 described below.

That is, according to some embodiments, the mounting/dismounting direction DE is a direction along the extension direction of the drive wire W. FIG. 11B is a cross-sectional view illustrating the relationship between the recess Wf and the protrusion 21d in the unlocked state of the joining section 21c.

The leaf spring 21ch of the joining section 21c includes a fixed portion 21cha fixed to the connecting base 21cb and a pressed portion 21chb in contact with the cam 21cc of the pressure member 21cp. The leaf spring 21ch has a first portion 21chd1 and a second portion 21chd2. When the catheter unit 100 is mounted to the base unit 200, the retained portion Wa is inserted between the first portion 21chd1 and the second portion 21chd2.

As illustrated in FIG. 11A, when the operating unit 400 is located at the dismount position, the pressure member 21cp is located at the retracted position which is retracted from the pressing position where the cam 21cc presses the leaf spring 21ch. In this state, the retained portion Wa is allowed to move in the extension direction of the drive wire W relative to the leaf spring 21ch, and the retained portion Wa is allowed to move in the dismounting direction Dd relative to the leaf spring 21ch.

More specifically, as illustrated in FIG. 11B, when the joining section 21c is the unlocked state, the protrusion 21d is retracted from the recess Wf, and the protrusion 21d is not in contact with (is separated from) the recess Wf.

As illustrated in FIG. 11B, the retained portion Wa has a first regulatory wall Wf1 on one side of the recess Wf and a second regulatory wall Wf2 on the other side of the recess Wf in the mounting/dismounting direction DE. That is, the recess Wf is formed between the first regulatory wall Wf1 and the second regulatory wall Wf2 in the mounting/dismounting direction DE.

As described below, when the protrusion 21d is inserted into the recess Wf, the retained portion Wa is fixed to the leaf spring 21ch. The first regulatory wall Wf1 and the second regulatory wall Wf2 extend in a direction that intersects the mounting/dismounting direction DE and are in contact with the protrusion 21d inserted into the recess Wf, thus regulating the movement of the retained portion Wa in the extension direction of the drive wire W or the mounting/dismounting direction DE.

According to some embodiments, each of the first regulatory wall Wf1 and the second regulatory wall Wf2 is inclined with respect to a direction perpendicular to the mounting/dismounting direction DE and the mounting/dismounting direction DE. The width of the recess Wf in the extension direction of the drive wire W or in the mounting/dismounting direction DE decreases from the upstream toward the downstream in a direction in which the protrusion 21d is inserted. This allows the protrusion 21d to be engaged with the recess Wf even if the center of the protrusion 21d and the center of the recess Wf are not aligned in the extension direction of the drive wire W or the mounting/dismounting direction DE.

As illustrated in FIG. 11A, the cam 21cc has a retaining surface 21cca and a pressure surface 21ccb. In the direction of radius of gyration of the pressure member 21cp, the retaining surface 21cca is disposed closer to a rotation center 21cpc of the pressure member 21cp than the pressure surface 21ccb.

As illustrated in FIG. 11A, in the dismountable state (the state in which the operating unit 400 is located at the dismount position), the leaf spring 21ch is retained at a position where the pressed portion 21chb is in contact with the retaining surface 21cca. A tooth Za1 of the internal gear 29 and a tooth Zb1 of the gear section 21cg are stopped, with a clearance La therebetween.

In the rotation direction of the operating unit 400, the direction in which the operating unit 400 moves from the dismount position to the release position and the fixation position is referred to as a lock direction (fixation direction), and the direction in which the operating unit 400 moves from the fixation position to the release position and the dismount position is referred to as a release direction. The operating unit 400 rotates from the release position in the release direction and moves to the dismount position. The operating unit 400 rotates from the release position in the lock direction and moves to the fixation position.

When the catheter unit 100 is mounted to the base unit 200 and the operating unit 400 is located at the dismount position, the joining section 21c is in the unlocked state, and the fixation of the drive wire W by the joining section 21c is released.

When the joining section 21c is in the unlocked state, the cam 21cc is located at the retracted position retracted from the pressing position (described below). At this time, the fixation of the retained portion Wa by the leaf spring 21ch and the protrusion 21d is released. When the joining section 21c is in the unlocked state, the protrusion 21d is retracted from the recess Wf. Therefore, when viewed in the extension direction of the drive wire W or the mounting/dismounting direction DE, the leaf spring 21ch and the protrusion 21d and the retained portion Wa do not overlap. More specifically, the protrusion 21d is located at a position where it does not overlap the recess Wf. In this state, the movement of the retained portion Wa in the extension direction of the drive wire W or the dismounting direction Dd is not regulated by the leaf spring 21ch and the protrusion 21d.

When the joining section 21c is in the unlocked state and if the catheter unit is moved in the dismounting direction Dd relative to the base unit 200, the retained portion Wa can be pulled out from between the first portion 21chd1 and the second portions 21chd2.

When the joining section 21c is in the unlocked state, it is desirable that the first portion 21chd1 and the second portion 21chd2 do not generate a force to tighten the retained portion Wa (generate a force of zero magnitude). When the joining section 21c is in the unlocked state, it is desirable that a gap be formed between at least one of the first portion 21chd1 and the second portion 21chd2 and the retained portion Wa.

FIG. 12 illustrates the internal gear 29 and the joining section 21c when the operating unit 400 is rotated from the dismount position in the lock direction. FIG. 12 illustrates the internal gear 29 and the joining section 21c when the operating unit 400 is located at the release position.

When the operating unit 400 that is located at the dismount position (FIG. 11A) is rotated in the lock direction, the internal gear 29 is rotated clockwise. The operating unit 400 is then located at the release position.

Even when the operating unit 400 is rotated, the entire catheter unit 100 (excluding the operating unit 400) is restricted from rotating relative to the base unit 200, because the key shaft 15 and the key receiving portion 22 are engaged. That is, the operating unit 400 can be rotated relative to the entire catheter unit 100 (excluding the operating unit 400) and the base unit 200 that are stopped.

Clockwise rotation of the internal gear 29 decreases the clearance between the tooth Za1 of the internal gear 29 and the tooth Zb1 of the gear section 21cg from the clearance La to a clearance Lb.

The tooth Zb2 of the gear section 21cg is disposed with a clearance Lz from the addendum circle of the tooth section 29g of the internal gear 29 (a dotted line). For this reason, the internal gear 29 can rotate without interfering with the tooth Zb2. The joining section 21c is maintained in the same state (the unlocked state) as the state illustrated in FIG. 11A.

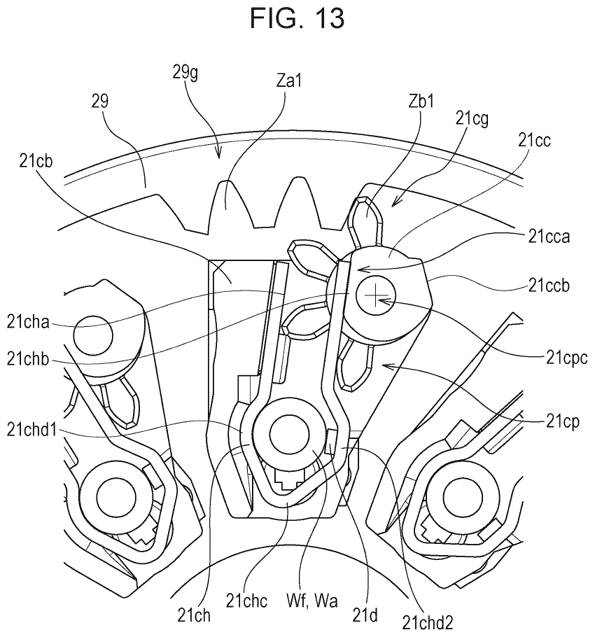

If the operating unit 400 is further rotated from the position illustrated in FIG. 12 in the lock direction, the internal gear 29 is further rotated clockwise. FIG. 13 illustrates the internal gear 29 and the joining section 21c at this time.

FIG. 13 illustrates the internal gear 29 and the joining section 21c when the operating unit 400 is rotated from the release position in the lock direction.

As illustrated in FIG. 13, when the operating unit 400 is rotated from the release position in the lock direction, the tooth Za1 of the internal gear 29 and the tooth Zb1 of the gear section 21cg are brought into contact. In contrast, the joining section 21c is in the same state as the state illustrated in FIGS. 11A and 12 and is maintained in the unlocked state.

FIG. 14 illustrates the pressure member 21cp being rotated by the rotation of the operating unit 400 in the lock direction.

As illustrated in FIG. 14, further rotation of the operating unit 400 in the lock direction from the position illustrated in FIG. 13 causes the internal gear 29 to further rotate clockwise.

Transition of the state of the internal gear 29 from the state illustrated in FIG. 13 to the state illustrated in FIG. 14 causes the internal gear 29 to rotate the gear section 21cg clockwise. As the gear section 21cg rotates, the retaining surface 21cca separates from the pressed portion 21chb, and the pressure surface 21ccb moves closer to the pressed portion 21chb. Then, the protrusion 21d moves closer to the recess Wf.

Thereafter, the pressed portion 21chb is moved to a position where a tooth Za3 of the internal gear 29 is separated from a tooth Zb3 of the gear section 21cg while being pressed by a corner portion 21ccb1 disposed at the edge of the pressure surface 21ccb. At this time, the protrusion 21d is inside of the recess Wf. In this state, the protrusion 21d and the leaf spring 21ch overlap each other as viewed in the extension direction of the drive wire W or the dismounting direction Dd. More specifically, the recess Wf and the protrusion 21d overlap at least partially.

When the tooth Za3 of the internal gear 29 is separated from the tooth Zb3 of the gear section 21cg, the transmission of the driving force from the internal gear 29 to the gear section 21cg ends. At this time, the cam 21cc is in a state in which the corner portion 21ccb1 receives a reaction force from the leaf spring 21ch.

In the direction of radius of gyration of the pressure member 21cp, the reaction force of the leaf spring 21ch acting on the corner portion 21ccb1 is exerted at a position away from the rotation center 21cpc of the pressure member 21cp and, thus, the pressure member 21cp rotates clockwise. At this time, the pressure member 21cp rotates in the same direction as the direction in which it is rotated by the internal gear 29 that rotates clockwise.

FIG. 15A illustrates the internal gear 29 and the joining section 21c when the operating unit 400 is located at the fixation position. FIG. 15B is a cross-sectional view illustrating the relationship between the recess Wf and the protrusion 21d in the locked state of the joining section 21c.

As illustrated in FIG. 15A, the pressure member 21cp receives the reaction force of the leaf spring 21ch and further rotates from the position illustrated in FIG. 14.

As illustrated in FIG. 15A, the pressure member 21cp is located at the pressing position and stops with the pressure surface 21ccb of the cam 21cc and the pressed portion 21chb of the leaf spring 21ch in surface contact. That is, the pressure surface 21ccb and the surface of the pressed portion 21chb are coplanar.

At this time, the joining section 21c is in the locked state. When the joining section 21c is in the locked state, the cam 21cc of the pressure member 21cp is located at the pressing position, and the pressure surface 21ccb presses against the pressed portion 21chb.

When the joining section 21c is in the locked state, the protrusion 21d is located inside of the recess Wf. In this state, the retained portion Wa and the protrusion 21d overlap as viewed in the extension direction of the drive wire W or the dismounting direction Dd. More specifically, the recess Wf and the protrusion 21d overlap at least partially. As a result, the retained portion Wa is fixed by the leaf spring 21ch and the protrusion 21d, and the retained portion Wa is restricted from moving in the mounting/dismounting direction DE relative to the joining section 21c.

Herein, the phrase “a portion overlaps another portion as viewed in a given direction” means that when one portion and another portion are projected onto a plane perpendicular to the given direction, a region where one portion overlaps the plane overlaps a region where the other portion overlaps the plane.

As can be seen from FIG. 10B, according to some embodiments, when the pressure member 21cp is located at the pressing position, the leaf spring 21ch and the retained portion Wa can be in contact with each other in the extension direction of the drive wire W (the same as the mounting/dismounting direction DE in FIG. 10B) in portions located on both sides of the recess Wf. The length in the extension direction of the region where the leaf spring 21ch and the retained portion Wa are in contact is greater than the length in the extension direction of the recess Wf. As a result, the leaf spring 21ch can stably support the retained portion Wa.

In addition, as illustrated in FIG. 15B, when the joining section 21c is in the locked state, that is, when the protrusion 21d is located inside of the recess Wf, the protrusion 21d is in contact with the first regulatory wall Wf1 and the second regulatory wall Wf2 of the retained portion Wa.

When the protrusion 21d is in contact with the first regulatory wall Wf1, the retained portion Wa of the drive wire W is restricted from moving in the mounting direction Da relative to the leaf spring 21ch and the protrusion 21d of the joining section 21c. When the protrusion 21d is in contact with the second regulatory wall Wf2, the retained portion Wa of the drive wire W is restricted from moving in the dismounting direction Dd relative to the leaf spring 21ch and the protrusion 21d of the joining section 21c.

In addition, as described above, the first regulatory wall Wf1 and the second regulatory wall Wf2 are disposed so that the recess Wf has a tapered shape. The leaf spring 21ch is elastically deformed when the protrusion 21d is in contact with the first regulatory wall Wf1 and the second regulatory wall Wf2. As a result, even if there are variations in the width of the protrusion 21d or the width of the recess Wf in the extension direction of the drive wire W or the mounting/dismounting direction DE, the protrusion 21d can still be in contact with the first regulatory wall Wf1 and the second regulatory wall Wf2.

According to some embodiments, both the first regulatory wall Wf1 and the second regulatory wall Wf2 are inclined with respect to a direction perpendicular to the mounting/dismounting direction DE and the mounting/dismounting direction DE. However, one of the first regulatory wall Wf1 and the second regulatory wall Wf2 may be inclined with respect to the direction perpendicular to the mounting/dismounting direction DE and the mounting/dismounting direction DE, and the other may be provided along the direction perpendicular to the mounting/dismounting direction DE.

In addition, according to some embodiments, the recess Wf has a tapered shape. However, the protrusion 21d may have a tapered shape. Alternatively, both the recess Wf and the protrusion 21d may have a tapered shape.

According to some embodiments, a bent portion 21chc that connects the first portion 21chd1 with the second portion 21chd2 is disposed between the first portion 21chd1 and the second portion 21chd2. The bent portion 21chc is disposed with a gap G from the retained portion Wa.

When the pressure member 21cp moves to the pressing position, the pressure member 21cp rotates in a direction in which a portion in contact with the leaf spring 21ch moves away from the bent portion 21chc. Therefore, the pressure member 21cp can rotate smoothly although receiving a reaction force from the leaf spring 21ch.

The material of the leaf spring 21ch can be resin or metal, but metal is desirable.

When the joining section 21c is in the locked state, it is restricted to pull out the retained portion Wa from between the first portion 21chd1 and the second portion 21chd2.

The tooth Za3 of the internal gear 29 and the tooth Zb4 of the gear section 21cg stop at positions so as to form a clearance Lc therebetween.

To release the fixation of the drive wire W and the joining section 21c, the operating unit 400 that is located at the fixation position is rotated in the release direction. At this time, the internal gear 29 rotates counterclockwise from the state illustrated in FIG. 15A. When the internal gear 29 rotates counterclockwise, the tooth Za3 of the internal gear 29 is brought into contact with the tooth Zb4 of the gear section 21cg, causing the pressure member 21cp to rotate counterclockwise.

Further counterclockwise rotation of the internal gear 29 releases the fixation of the drive wire W by the joining section 21c. The operation performed by the internal gear 29 and the pressure member 21cp at this time is the reverse of the operation described above. That is, the fixation of the drive wire W by the joining section 21c is released by the reverse operation of the above-described operation performed when the drive wire W is fixed by the joining section 21c.

The above-described operation is performed on each of the first to ninth joining sections (21c11 to 21c33). That is, in the process of moving the operating unit 400 from the dismount position to the fixation position, the movement (rotation) of the operating unit 400 causes the first to ninth joining sections (21c11 to 21c33) to enter the locked state from the unlocked state. In the process of moving the operating unit 400 from the fixation position to the dismount position, the movement (rotation) of the operating unit 400 causes the first to ninth joining sections (21c11 to 21c33) to enter the unlocked state from the locked state. That is, the user can switch between the unlocked state and the locked state of the plurality of joining sections by operating the single operating unit 400.

That is, an operating unit need not be provided for each of the plurality of joining sections to switch between the unlocked state and the locked state and, thus, the user need not operate each of the operating units. Therefore, the user can easily mount and dismount the catheter unit 100 to and from the base unit 200. Furthermore, the medical device 1 can be simplified.

The state in which the first to ninth drive wires (W11 to W33) are fixed by the first to ninth joining sections (21c11 to 21c33), respectively, is referred to as a first state. The state in which fixation of the first to ninth drive wires (W11 to W33) by the first to ninth joining sections (21c11 to 21c33), respectively, is released is referred to as a second state.

The first state and the second state are switched in conjunction with the movement of the operating unit 400. That is, the first state and the second state are switched in conjunction with the movement of the operating unit 400 between the dismount position and the fixation position.

The internal gear 29 is configured to interlock with the operating unit 400. According to some embodiments, the joint 28 functions as a transmission member for interlocking the operating unit 400 with the internal gear 29. The internal gear 29 and joint 28 function as an interlock unit to interlock with the operating unit 400 so that the first state and the second state are switched in conjunction with the movement of the operating unit 400.

More specifically, the internal gear 29 and the joint 28 move a portion of the leaf spring 21ch (the pressed portion 21chb) relative to the retained portion Wa in conjunction with the movement of the operating unit 400 with the catheter unit 100 mounted to the base unit 200. The movement of the pressed portion 21chb can switch between the locked state and the unlocked state of the joining section 21c.

The internal gear 29 may be configured to be moved directly by the operating unit 400. In this case, the internal gear 29 functions as the interlock unit.

As described above, when the operating unit 400 is located at the fixation position, the pressure member 21cp is located at the pressing position where the cam 21cc presses against the leaf spring 21ch. When the pressure member 21cp is located at the pressing position and the joining section 21c is in the locked state, the protrusion 21d is inserted into and located inside the recess Wf. In this state, the protrusion 21d and the leaf spring 21ch overlap as viewed in the extension direction of the drive wire W or the mounting/dismounting direction DE (the dismounting direction Dd and the mounting direction Da). More specifically, the recess Wf and the protrusion 21d overlap at least partially. As a result, the retained portion Wa is restricted from moving relative to the leaf spring 21ch in the extension direction of the drive wire W or the mounting/dismounting direction DE (the dismounting direction Dd and the mounting direction Da).

That is, according to the configuration of some embodiments, when the retained portion Wa attempts to move in the extension direction of the drive wire W or the mounting/dismounting direction DE, the retained portion Wa interferes with the protrusion 21d that is part of the elastic member, thus regulating the movement of the retained portion Wa. Therefore, the retained portion Wa can be stably fixed to the joining section 21c, regardless of the magnitude of the force with which the leaf spring 21ch tightens the retained portion Wa or the coefficient of friction between the retained portion Wa and the leaf spring 21ch.

When the joining section 21c moves in the dismounting direction Dd to push the drive wire W or when the joining section 21c moves in the mounting direction Da to pull the drive wire W, the joining section 21c can move the retained portion Wa more accurately.

That is, unlike the configuration in which the first portion 21chd1 and the second portion 21chd2 retain the retained portion Wa by the force to tighten the retained portion Wa, the pressure member 21cp need not press against the leaf spring 21ch with a strong force. For this reason, the force that the pressure member 21cp receives from the leaf spring 21ch when rotating the pressure member 21cp can also be reduced. As a result, the force required to operate the operating unit 400 can be reduced.

According to some embodiments, the direction in which the bent portion 21chc of the leaf spring 21ch extends is the extension direction of the drive wire W or the dismounting direction Dd. Therefore, deformation of the leaf spring 21ch can be reduced when the retained portion Wa interferes with part of the leaf spring 21ch which is the elastic member and, thus, the retained portion Wa can be stably fixed to the joining section 21c.

### Movement of Operating Unit

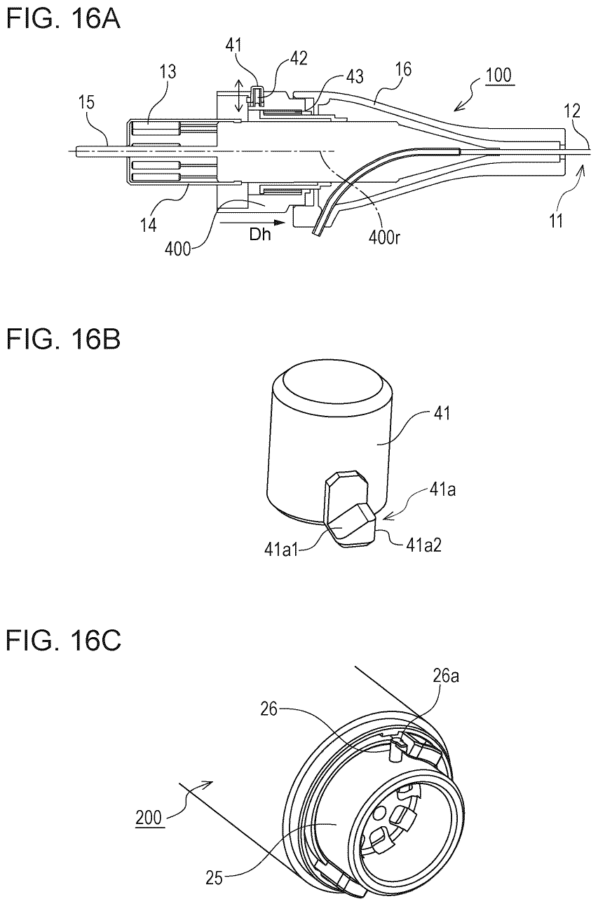

The movement of the operating unit 400 is described below with reference to FIGS. 16A to 16C, FIGS. 17A to 17C, and FIGS. 18A to 18C.

According to some embodiments, the operating unit 400 is configured to be movable between the dismount position, the release position, and the fixation position when the catheter unit 100 is mounted to the base unit 200. The release position is located between the dismount position and the fixation position.

According to some embodiments, the first state and the second state are switched in conjunction with the movement of the operating unit 400 between the release position and the fixation position.

According to some embodiments, the operating unit 400 can move between the dismount position and the fixation position by moving in a direction different from the mounting/dismounting direction DE. The operating unit 400 moves between the dismount position and the fixation position by moving in a direction that intersects (preferably orthogonal to) the mounting/dismounting direction DE. According to some embodiments, the operating unit 400 rotates about the rotation axis 400r extending in the mounting/dismounting direction DE to move between the dismount position and the fixation position. Thus, excellent operability is provided for a user when the user operates the operating unit 400.

FIGS. 16A to 16C illustrate the catheter unit 100 and the base unit 200. FIG. 16A is a cross-sectional view of the catheter unit 100. FIG. 16B is a perspective view of a button 41. FIG. 16C is a perspective view of the base unit 200.

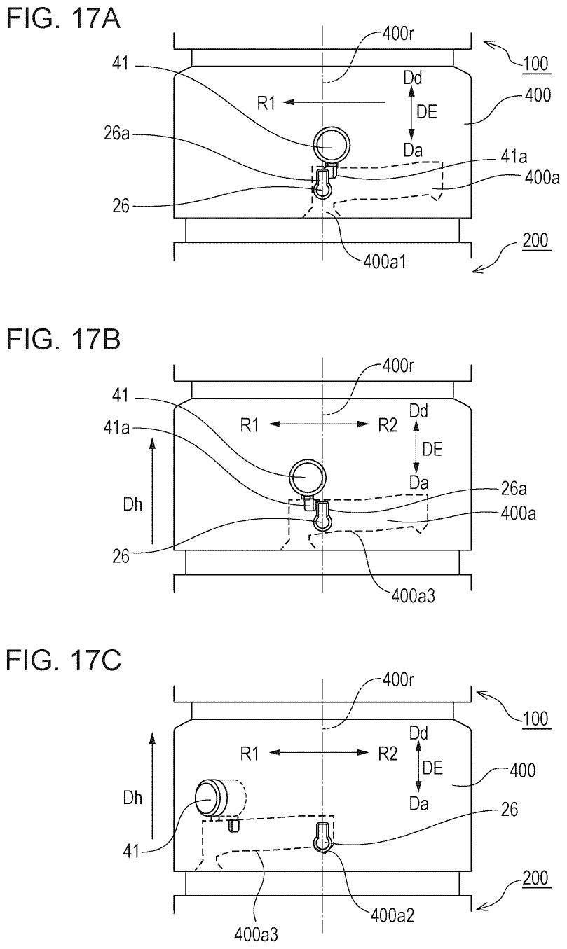

FIGS. 17A to 17C illustrate the movement of the operating unit 400. FIG. 17A illustrates the operating unit 400 located at the dismount position. FIG. 17B illustrates the operating unit 400 located at the release position. FIG. 17C illustrates the operating unit 400 located at the fixation position.

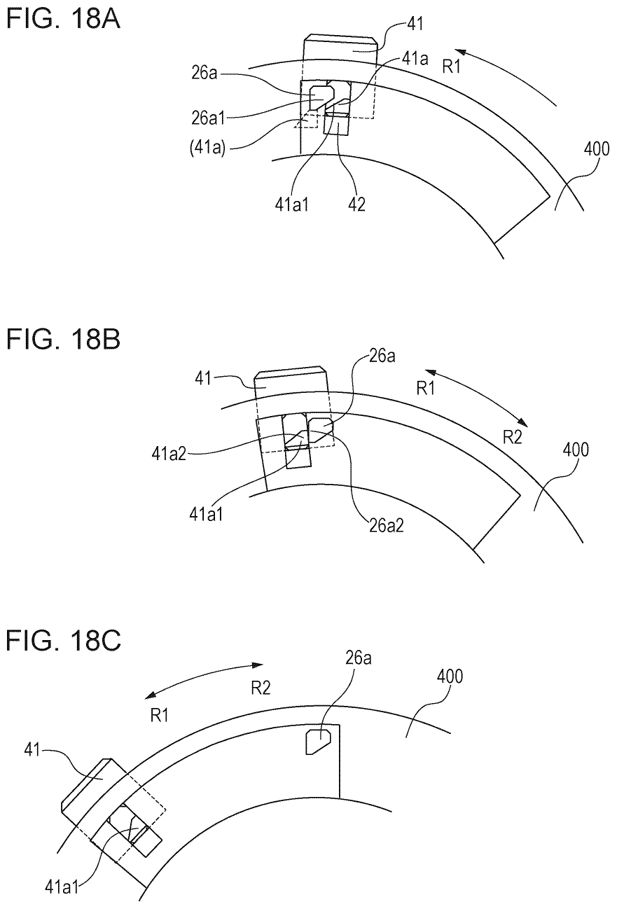

FIGS. 18A to 18C are cross-sectional views illustrating the movement of the operating unit 400. FIG. 18A is a cross-sectional view of the operating unit 400 located at the dismount position. FIG. 18B is a cross-sectional view of the operating unit 400 located at the release position. FIG. 18C is a cross-sectional view of the operating unit 400 located at the fixation position.

When the operating unit 400 is located at the fixation position, the joining section 21c is in the locked state, and the retained portion Wa of the drive wire W is fixed to a corresponding joining section 21c (refer to FIG. 15A).

When the operating unit 400 is located at the release position, the pressure member 21cp is located at the retracted position, the joining section 21c is in the unlocked state, and the lock between the retained portion Wa of the drive wire W and the joining section 21c is released (refer to FIG. 12). At this time, the connection between the drive wire W and the wire drive unit 300 is disconnected. Therefore, when the catheter 11 is subjected to an external force, the bending section 12 can be bent freely without resistance by the wire drive unit 300.

When the operating unit 400 is located at the dismount position, the catheter unit 100 is allowed to be dismounted from the base unit 200. The catheter unit 100 can be mounted to the base unit 200 when the operating unit 400 is located at the dismount position. When the operating unit 400 is located at the dismount position, the joining section 21c is in the unlocked state, and the lock between the retained portion Wa of the drive wire W and the joining section 21c is released (refer to FIG. 11A).

As illustrated in FIG. 16A, the catheter unit 100 includes an operating unit biasing spring 43 that biases the operating unit 400, the button 41 serving as a moving member, and a button spring 42 that biases the button 41.

According to some embodiments, the operating unit biasing spring 43 is a compression spring. The operating unit 400 is biased by the operating unit biasing spring 43 in a direction Dh closer to the proximal-end cover 16.

According to some embodiments, the button 41 and the button spring 42 are provided in the operating unit 400. The button 41 and the button spring 42 move together with the operating unit 400 when the operating unit 400 is moved to the dismount position, the release position, or the fixation position.

The button 41 is movable relative to the operating unit 400 in a direction that intersects the rotation axis 400r of the operating unit 400. The button 41 is biased toward the outside of the catheter unit 100 (in a direction away from the rotation axis 400r) by the button spring 42.

As described below, the button 41 regulates the movement of the operating unit 400 from the release position to the dismount position. By moving the button 41 relative to the operating unit 400, the operating unit 400 is allowed to move from the release position to the dismount position.

The button 41 has a button protrusion (regulated portion) 41a. The button protrusion 41a has a button slope face 41a1 and a regulated surface 41a2.

The base unit 200 includes the base frame 25. The base frame 25 includes the locking shaft 26. The locking shaft 26 has the locking protrusion (regulatory portion) 26a.

According to some embodiments, the locking shaft 26 is provided in plurality (two according to some embodiments). All of the locking shafts 26 may have the locking protrusions 26a, or some of the locking shafts 26 may have the locking protrusions 26a.

As illustrated in FIG. 9, FIG. 17A, FIG. 17B, and FIG. 17C, a locking groove 400a that engages with the locking shaft 26 is provided inside of the operating unit 400. The locking groove 400a extends in a direction different from the mounting/dismounting direction DE. According to some embodiments, the locking groove 400a extends in the rotation direction of the operating unit 400. That is, the locking groove 400a extends in a direction that intersects (that is orthogonal to) the mounting/dismounting direction DE.

When a plurality of locking shafts 26 are provided, the locking groove 400a is provided for each of the plurality of locking shafts 26.

As illustrated in FIG. 17A, when the catheter unit 100 is mounted to the base unit 200, the locking shaft 26 is engaged with the locking groove 400a via an entrance 400al of the locking groove 400a.

At this time, the operating unit 400 is located at the dismount position, and the joining section 21c is in the unlocked state (refer to FIG. 11A). Therefore, the fixation of the first to ninth drive wires (W11 to W33) by the first to ninth joining sections (21c11 to 21c33), respectively, is released. As illustrated in FIG. 18A, the button protrusion 41a and the locking protrusion 26a face each other.

When the operating unit 400 is rotated in a lock direction R1 with the operating unit 400 located at the dismount position, the slope face 41al of the button protrusion 41a is in contact with a slope face 26al of the locking protrusion 26a. The button 41 moves toward the inside of the operating unit 400 (in the direction closer to the rotation axis 400r) against the biasing force of the button spring 42. The button protrusion 41a then passes over the locking protrusion 26a, and the operating unit 400 moves to the release position (refer to FIG. 18B).

At this time, the joining section 21c is in the unlocked state (refer to FIG. 12). Therefore, the fixation of the first to ninth joining sections (21c11 to 21c33) by the first to ninth drive wires (W11 to W33), respectively, is released.

According to some embodiments, the operating unit 400 is allowed to move from the dismount position to the release position without the button 41 being operated. That is, the user need not operate the button 41 when moving the operating unit 400 from the dismount position to the release position.

When the operating unit 400 is located at the release position and if the operating unit 400 is rotated in the lock direction R1, the operating unit 400 moves to the fixation position. When the operating unit 400 is located at the fixation position, a positioning portion 400a2 of the locking groove 400a is located at a position corresponding to the locking shaft 26. The operating unit 400 is biased in the direction Dh closer to the proximal-end cover 16 by the operating unit biasing spring 43. As a result, the positioning portion 400a2 is engaged with the locking shaft 26.

In the process of moving the operating unit 400 from the release position to the fixation position, the retained portion Wa of the drive wire W is fixed to the joining section 21c, as described above.

When the operating unit 400 is located at the fixation position, the joining section 21c is in the locked state (refer to FIG. 15A). Therefore, the first to ninth drive wires (W11 to W33) are fixed to the first to ninth joining sections (21c11 to 21c33), respectively. In this state, the driving force from the wire drive unit 300 can be transmitted to the bending drive unit 13. That is, the driving forces from the first to ninth drive sources (M11 to M33) can be transmitted to the first to ninth drive wires (W11 to W33) via the first to ninth joining sections (21c11 to 21c33), respectively.

When the operating unit 400 is located at the release position, a wall 400a3 that forms the locking groove 400a is located upstream of the locking shaft 26 in the dismounting direction Dd of the catheter unit 100. When the operating unit 400 is located at the fixation position, the positioning portion 400a2 is located upstream of the locking shaft 26 in the dismounting direction Dd. As a result, when the operating unit 400 is located at the release position or at the fixation position, the catheter unit 100 is restricted from being dismounted from the base unit 200. In contrast, when the operating unit 400 is located at the dismount position, the entrance 400al of the locking groove 400a is located upstream of the locking shaft 26 in the dismounting direction Dd. As a result, the catheter unit 100 is allowed to be dismounted from the base unit 200.

When the operating unit 400 is located at the fixation position and if the operating unit 400 is rotated in a release direction R2, the operating unit 400 is located at the release position. In the process of moving the operating unit 400 from the fixation position to the release position, the retained portion Wa of the drive wire W is released from the joining section 21c, as described above.

When the operating unit 400 is located at the release position, the regulated surface 41a2 of the button protrusion 41a is in contact with the regulating surface 26a2 of the locking protrusion 26a (refer to FIG. 18B). In this state, the operating unit 400 is restricted from rotating in the release direction R2. In addition, the catheter unit 100 is restricted from being dismounted from the base unit 200.

When the operating unit 400 is located at the release position and if the user pushes the button 41 toward the inside of the operating unit 400, the regulated surface 41a2 is separated from a regulating surface 26a2, and the button protrusion 41a passes over the locking protrusion 26a. As a result, the operating unit 400 is allowed to rotate in the release direction R2 and, thus, the operating unit 400 can move from the release position to the dismount position.

When the operating unit 400 is located at the dismount position, the joining section 21c enters the unlocked state. Therefore, the load acting on the drive wire W (for example, the resistance received by the joining section 21c) can be reduced when the catheter unit 100 is dismounted from or mounted to the base unit 200. As a result, the user can easily mount and dismount the catheter unit 100.

When the operating unit 400 is located at the release position, the catheter unit 100 is restricted from being dismounted from the base unit 200, and the joining section 21c enters the unlocked state. As described above, when the joining section 21c is in the unlocked state, the connection between the drive wire W and the wire drive unit 300 is disconnected, the bending section 12 can be bent freely without receiving resistance by the wire drive unit 300.

The user can stop driving of the catheter 11 using the wire drive unit 300 by positioning the operating unit 400 at the release position while the catheter 11 is inserted inside the subject. Furthermore, since the catheter unit 100 is restricted from being dismounted from the base unit 200, the user can hold the base unit 200 and pull out the catheter 11 from the inside of the subject.

According to the configuration of some embodiments, if the button 41 is not operated, the operating unit 400 is restricted from moving from the release position to the dismount position. Therefore, when the user moves the operating unit 400 from the fixation position to the release position, the user is prevented from accidentally moving the operating unit 400 to the dismount position.

According to some embodiments, the number of locking protrusions 26a and the number of the buttons 41 are one each. However, the medical device 1 may include more than one locking protrusion 26a and more than one button 41.

### MODIFICATIONS

According to the above-described embodiment, the retained portion Wa has the recess Wf, and the leaf spring 21ch has the protrusion 21d. However, the retained portion Wa may have a protrusion, and the leaf spring 21ch may have a shape of a hole, a groove, or the like into which the protrusion is to be inserted.

As described above, the present invention uses an elastic member to stably fix a linear member connected with a bending section to a joining section connected to a drive source.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical device comprising:
a bending section;
a linear member connected to the bending section and configured to move in an extension direction so as to bend the bending section, wherein the linear member includes an engaged portion;
a joining section connected to a drive source, wherein the joining section includes an elastic member configured to retain the engaged portion and a first rotating body rotatable to a pressing position where the elastic member is pressed or a retracted position that is retracted from the pressing position; and
a second rotating body movable to a first position where the first rotating body is located at the pressing position or a second position where the first rotating body is located at the retracted position,
wherein when the first rotating body is located at the pressing position, the engaged portion and the elastic member overlap each other as viewed in the extension direction so that the engaged portion is restricted from moving relative to the elastic member in the extension direction.

2. The medical device according to claim 1, wherein the elastic member includes an insertion portion, and the engaged portion includes an inserted portion, and
wherein when the first rotating body is located at the pressing position, the insertion portion is inserted into the inserted portion, and the inserted portion overlaps the insertion portion as viewed in the extension direction.

3. The medical device according to claim 2, wherein when the pressure member is located at the pressing position, the elastic member is contactable with parts of a retained portion located on both sides of the inserted portion in the extension direction.

4. The medical device according to claim 2 wherein when the pressure member is located at the pressing position, a length of a region in which the elastic member is in contact with a retained portion in the extension direction is greater than a length of the inserted portion in the extension direction.

5. The medical device according to claim 2, wherein the elastic member includes a leaf spring, and the leaf spring is bent so as to form the insertion portion.

6. The medical device according to claim 1, wherein when the second rotating body is located at the second position and the first rotating body is located at the retracted position, a retained portion is allowed to move relative to the elastic member in a dismounting direction in which the retained portion is dismounted from the elastic member, and
wherein when the first rotating body is located at the pressing position, the engaged portion and the elastic member overlap each other as viewed in the dismounting direction so that the engaged portion is restricted from moving relative to the elastic member in the dismounting direction.

7. The medical device according to claim 6, further comprising:
a bending body unit including the bending section and the linear member; and
a joining unit including the joining section,
wherein the bending body unit is dismountably mounted to the joining unit.

8. The medical device according to claim 7, wherein the second rotating body is movable to a third position between the first position and the second position,
wherein when the second rotating body is located at the second position, the bending body unit is allowed to be mounted to and dismounted from the joining unit, and
wherein when the second rotating body is located at the third position, the first rotating body is located at the retracted position, and the bending body unit is restricted from being mounted to and dismounted from the joining unit.
